(19) United States
(12) Patent Application Publication
(10) Pub. No.: US 2006/0029668 A1
(43) Pub. Date: Feb. 9, 2006

(54) Sustained release L-arginine formulations and methods of manufacture and use

(76) Inventor: Eyal S. Ron, Lexington, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/125,308

(22) Filed: May 9, 2005

Related U.S. Application Data
(63) Continuation-in-part of application No. 11/108,054, filed on Apr. 15, 2005.
Application No. 11/108,054 is a continuation-in-part of application No. PCT/US03/33931, filed on Oct. 24, 2003.
(60) Provisional application No. 60/512,035, filed on Oct. 17, 2003.
(60) Provisional application No. 60/507,312, filed on Sep. 29, 2003.
(60) Provisional application No. 60/421,258, filed on Oct. 24, 2002.

(51) Int. Cl. A61K 31/198; A61K 9/22

(52) U.S. Cl. 424/468; 514/565

(57) ABSTRACT

The present invention provides methods and formulations for the treatment and prevention of cerebrovascular and cardiovascular diseases and disorders. The present invention is based, at least in part, on the discovery that administering to a subject a formulation comprising an agonist of endothelial nitric oxide synthase (eNOS), such as an HMG-CoA reductase inhibitor, and a formulation comprising a precursor of NO, such as L-arginine, may be used to treat or prevent cerebrovascular and/or cardiovascular diseases or disorders.

### RELATED APPLICATIONS

This application is a continuation in part of U.S. application Ser. No. 11/108,054, entitled “Sustained Release L-Arginine Formulations and Methods of Manufacture and Use” filed Apr. 15, 2005 which is a continuation in of U.S. application No. PCT/US03/03393 1, entitled “Sustained Release L-Arginine Formulations and Methods of Manufacture and Use” filed Oct. 24, 2003 which claims priority to U.S. Provisional Patent Application Ser. No. 60/421,258, entitled “Methods and Compositions for the Treatment of Cerebrovascular and Cardiovascular Diseases and Disorders” filed Oct. 24, 2002, U.S. Provisional Patent Application Ser. No. 60/507,312, entitled “Methods and Compositions for the Treatment of Cerebrovascular and Cardiovascular Diseases and Disorders” filed Sep. 29, 2003, and U.S. Provisional Patent Application Ser. No. 60/512,035, entitled “Sustained Release L-Arginine Formulations and Methods of Manufacture and Use” filed Oct. 17, 2003; the entire contents of each of the aformentioned applications are hereby incorporated herein by reference in their entirety.

### BACKGROUND OF THE INVENTION

A family of enzymes called nitric oxide synthases (NOS) synthesize nitric oxide (NO), an important biological second messenger, from L-arginine. There are several distinct isoforms of NOS including constitutive NOS (cNOS) and inducible NOS (iNOS). There are two different kinds of cNOS: endothelial NOS (eNOS) and neuronal NOS (nNOS). eNOS is involved in the regulation of smooth muscle relaxation, blood pressure lowering, and inhibition of platelet aggregation. eNOS resides in endothelial cells and releases NO over short periods in response to receptor-mediated increases in cellular Ca²⁺. Michel et al., “Nitric oxide synthases: which, where, how, and why?,” J. Clin. Invest 100: 2146-2152 (1997). nNOS is important for long-term potentiation, and is responsible for the Ca²⁺ dependent release from neurons. iNOS acts in host defense, is generated by activated macrophage cells during an immune response, is induced in vascular smooth muscle cells (e.g., by various cytokines, microbial products, and/or bacterial endotoxins), and once expressed, synthesizes NO for long periods of time.

Formation of nitric oxide by cNOS in endothelial cells is thought to play an important role in normal blood pressure regulation, prevention of endothelial dysfunction such as hyperlipidemia, arteriosclerosis, thrombosis, and restenosis. Functionally, cNOS, which is the predominant synthase present in brain and endothelia, is active under basal conditions and can be further stimulated by increases in intracellular calcium that occur in response to receptor-mediated agonists or calcium ionophores. cNOS appears to be the “physiological” form of the enzyme and plays a role in a diverse group of biological processes. In vitro studies suggest that the activity of NOS can be regulated in a negative feedback manner by nitric oxide itself. In cardiocerebrorenovascular circulation, the primary target for constitutively produced NO is believed to be soluble guanylate cyclase located in vascular smooth muscle, the myocardium (myocytes) and coronary vascular smooth muscle.

In contrast to cNOS, the inducible, calcium-independent isoform, iNOS was initially only described in macrophages. It is now known that induction of nitric oxide synthase can occur in response to appropriate stimuli in many other cell types. This induction occurs both in cells that normally do not express a constitutive form of nitric oxide synthase, such as vascular smooth muscle cells, as well as in cells such as those of the myocardium that express considerable levels of the constitutive isoform.

iNOS exhibits negligible activity under basal conditions, but in response to factors such as lipopolysaccharide and certain cytokines, expression occurs over a period of hours. The induced form of the enzyme produces much greater amounts of NO than the constitutive form, and induced NOS appears to be the “pathophysiological” form of the enzyme because high concentrations of iNOS produced NO can be toxic to cells. Induction of iNOS can be inhibited by glucocorticoids and some cytokines. Relatively little is known about post-transcriptional regulation of iNOS. Cytotoxic effects due to NO are probably largely independent of guanylate cyclase and cyclic GMP formation. Most of the research in this area has focused on the stimulation of iNOS inhibitors using various derivatives of L-arginine.

NO is a relatively stable free radical synthesized from molecular oxygen and the guanidino nitrogen of L-arginine in a reaction catalyzed by NOS. This enzyme is found in many tissues and cell types including neurons, macrophages, hepatocytes, smooth muscle cells, endothelial cells of the blood vessels, and epithelial cells of the kidney. NO acts near its point of release, entering the target cell and activating the cytosolic enzyme guanylate cyclase, which catalyzes the formation of the second messenger cyclic GMP (cGMP). Within seconds of the formation of NO, it undergoes oxidation to nitrite or nitrate. David L. Nelson, Michael M. Cox, Lehninger Principles of Biochemistry, p. 892, 3rd ed. Worth Publishers, 2000.

In response to a variety of vasoactive agents and even physical stimuli, the endothelial cells release a short-lived vasodilator called endothelium derived relaxing factor (EDRF) (also referred to as endothelium derived nitric oxide (EDNO)). Products of inflammation and platelet aggregation such as serotonin, histamine, bradykinin, purines, and thrombin exert all or part of their action by stimulating the release of NO. Endothelial cell-dependent mechanisms of relaxation are important in a variety of vascular beds, including the coronary circulation. Hobbs et al., Annu. Rev. Pharmacol. Toxicol. 39: 191-220 (1999). NO diffuses readily to the underlying smooth muscle and induces relaxation of vascular smooth muscle by activating guanylate cyclase, which increases cGMP concentrations.

NO is responsible for the endothelium dependent relaxation and activation of soluble guanylate cyclase, neurotransmission in the central and peripheral nervous systems, and activated macrophage cytotoxicity. In the vasculature, EDNO has several actions among which are the inhibition of platelet aggregation, adhesion of inflammatory cells, and the proliferation of smooth muscle cells. In particular, EDNO is an important regulator of vascular tone. Also, flow dependent dilation, a commonly used index of endothelial function, is largely mediated by NO.

The mechanism for the regulation of vascular tone by NO is initiated by stimuli, such as acetylcholine, bradykinin, shear stress, etc., on the endothelial cells lining the vasculature. NO is produced from L-arginine through the catalytic activity of eNOS contained in these endothelial cells. The NO produced leaves the endothelial cells and stimulates the guanylate cyclase activity in the adjoining smooth muscle cells. Activation of guanylate cyclase increases the level of cGMP and causes the smooth cells to relax, thus dilating the vessel and increasing the blood flow. Moncada et al., New Eng. J. Med. 329: 2002-2012 (1993); Vallance et al., J. Royl. Coll. Physician London 28: 209-219 (1994).

### SUMMARY OF THE INVENTION

The present invention provides methods for the treatment and prevention of vascular diseases and disorders including, but not limited to, cardiovascular, cerebrovascular and peripheral vascular diseases and disorders. The present invention is based, at least in part, on the discovery that the coadministration of an HMG-CoA reductase inhibitor and a sustained release formulation of L-arginine has a synergistic effect in the treatment and prevention of vascular diseases and disorders, and, in particular, in lowering cholesterol and triglycerides. Moreover, the invention provides a sustained release formulation of L-arginine and methods of manufacture that render a composition with an optimal release profile. Furthermore, the formulation and methods of manufacture render a composition that is conveniently compressible, but not excessively friable.

In one aspect, the invention provides a method for lowering cholesterol in a subject, including administering to a subject an HMG-CoA reductase inhibitor and a sustained release formulation comprising L-arginine. In various embodiments, the method lowers total cholesterol, low density lipoprotein (LDL) cholesterol and/or triglyceride levels. Moreover, the method increases high density lipoprotein (HDL) cholesterol. Furthermore, the method lowers total cholesterol, LDL cholesterol and/or triglyceride levels, and/or increases HDL cholesterol to a greater extent than merely administering HMG-CoA reductase inhibitor without L-arginine.

In another aspect, the present invention provides a method for increasing nitric oxide production in a subject with elevated asymmetrical dimethylarginine (ADMA) by administering to the subject an HMG-CoA reductase inhibitor and L-arginine. In yet another aspect, the present invention provides a method for increasing vasodilation in a subject with elevated asymmetrical dimethylarginine (ADMA) by administering to the subject an HMG-CoA reductase inhibitor and L-arginine. In various embodiments of these aspects of the invention, L-arginine is present as a sustained release formulation. In other embodiments, the HMG-CoA reductase inhibitor is simvastatin. In certain embodiments, the subject may have endothelial dysfunction. In other embodiments of these aspects of the invention, the method increases endothelial function.

In another aspect, the present invention provides a method for increasing nitric oxide (NO) production in a subject with elevated asymmetrical dimethylarginine (ADMA) by administering L-arginine to the subject, wherein the L-arginine overcomes the inhibitory effect of ADMA. In yet another aspect, the present invention provides a method for increasing vasodilation in a subject with elevated asymmetrical dimethylarginine (ADMA), by administering L-arginine to the subject, wherein the L-arginine overcomes the inhibitory effect of ADMA. In various embodiments of these aspects of the invention, HMG-CoA reductase inhibitor (e.g., simvastatin) is coadministered with the L-arginine. In certain embodiments, the L-arginine is present as a sustained release formulation. In other embodiments of these aspects of the invention, the method increases endothelial function.

In another aspect, the invention provides a sustained release L-arginine composition including about 25% to about 75% by weight of L-arginine or a pharmaceutically acceptable salt thereof; about 0.5% to about 5% by weight of polyvinylpyrrolidone; about 5% to about 40% by weight of hydroxypropyl methylcellulose; about 2% to about 20% by weight of microcrystalline cellulose; less than about 3% by weight of silicon dioxide; and less than about 3% by weight of magnesium stearate. In a particular embodiment, the composition includes about 50% by weight of L-arginine monohydrochloride, where the L-arginine is L-arginine monohydrochloride; between about 3% and about 4% by weight of polyvinylpyrrolidone; about 35% by weight of hydroxypropyl methylcellulose; about 10% by weight of microcrystalline cellulose; less than about 1% by weight of colloidal silicon dioxide, where the silicon dioxide is colloidal silicon dioxide; and less than about 1% by weight of magnesium stearate.

In another aspect, the invention provides a method for making a sustained release composition of L-arginine, including granulating L-arginine with a granulating agent to form granules; wet milling the granules; drying the granules; dry milling the granules; and blending the granules with at least one sustained release agent. In various embodiments, the blending step may include pre-blending, blending and final blending the granules. In another embodiment, the method may include dry mixing the L-arginine with a binder prior to the granulating step. The binder may be polyvinylpyrrolidone.

In a particular embodiment of this aspect of the invention, the method includes granulating L-arginine, where L-arginine is about 50% by weight of the sustained release formulation, with granulating agent including polyvinylpyrrolidone, where polyvinylpyrrolidone is between about 3% and about 4% by weight of the sustained release formulation; wet milling the granules; drying the granules; dry milling the granules; and blending the granules with hydroxypropyl methylcellulose, where the hydroxypropyl methylcellulose is about 35% by weight of the sustained release formulation. The method may further include blending the granules with microcrystalline cellulose, colloidal silicon dioxide and magnesium stearate, where the microcrystalline cellulose is about 10% by weight of the sustained release formulation, where colloidal silicon dioxide is less than about 1% of the sustained release formulation, and where the magnesium stearate comprises less than about 1% by weight of the sustained release formulation.

In another aspect, the invention provides a food bar including a sustained release formulation of L-arginine (e.g., sustained release granulars of L-arginine) for use in treating or preventing a vascular disease or disorder. The food bar may also include an HMG-CoA reductase inhibitor (e.g., simvastatin). In various embodiments, the food bar lowers cholesterol, lowers C-reactive protein, can treat or prevent Alzheimer's Disease, and/or can treat or prevent intermittent claudication.

In another aspect, the invention provides a method for preventing or treating a vascular disease or disorder in a subject, including administering to a subject a food bar with a sustained release formulation of L-arginine. In yet another aspect, the invention provides a method for lowering cholesterol in a subject, including administering to a subject a food bar with a sustained release formulation of L-arginine. In yet another aspect, the invention provides a method for increasing nitric oxide in a subject, including administering to a subject a food bar with a sustained release formulation of L-arginine. In a further aspect, the invention provides a method for increasing vasodilation in a subject, including administering to a subject a food bar with a sustained release formulation of L-arginine. In another aspect, the invention provides a method for treating or preventing Alzheimer's Disease in a subject, including administering to a subject a food bar with a sustained release formulation of L-arginine. In yet another aspect, the invention provides a method for treating or preventing intermittent claudication in a subject, including administering to a subject a food bar with a sustained release formulation of L-arginine. In yet another aspect, the invention provides a method for lowering C-reactive protein in a subject, including administering to a subject a food bar with a sustained release formulation of L-arginine. In certain embodiments of the preceding aspects, the food bar may also include an HMG-CoA reductase inhibitor (e.g., simvastatin).

In another aspect, the invention provides a method for lowering cholesterol in a subject, including administering to a subject a sustained release formulation of L-arginine. In various embodiments, the method may lower total cholesterol, low density lipoprotein (LDL) cholesterol, and/or triglycerides, and/or increase high density lipoprotein (HDL) cholesterol in the subject. In another aspect, the invention provides a method for treating or preventing Alzheimer's disease, including administering to a subject a sustained release formulation of L-arginine. In yet another aspect, the invention provides a method for treating or preventing intermittent claudication, including administering to a subject a sustained release formulation of L-arginine. In yet another aspect, the invention provides a method for lowering C-reactive protein, including administering L-arginine (e.g., sustained release L-arginine) to a subject. In certain embodiments of the preceding aspects of the invention, the sustained release formulation includes about 25% to about 75% by weight of L-arginine or a pharmaceutically acceptable salt thereof; about 0.5% to about 5% by weight of polyvinylpyrrolidone; about 5% to about 40% by weight of hydroxypropyl methylcellulose; about 2% to about 20% by weight of microcrystalline cellulose; less than about 3% by weight of silicon dioxide; and less than about 3% by weight of magnesium stearate. In a particular embodiment, the sustained release formulation includes about 50% by weight of L-arginine monohydrochloride, where the L-arginine is L-arginine monohydrochloride; between about 3% and about 4% by weight of polyvinylpyrrolidone; about 35% by weight of hydroxypropyl methylcellulose; about 10% by weight of microcrystalline cellulose; less than about 1% by weight of colloidal silicon dioxide, where the silicon dioxide is colloidal silicon dioxide; and less than about 1% by weight of magnesium stearate.

In various other aspects, the present invention provides a method for treating or preventing a vascular disease or disorder, a method for treating or preventing atherosclerosis, a method for increasing vasodilation, and/or a method for increasing nitric oxide production, including administering to a subject a sustained release formulation including about 25% to about 75% by weight of L-arginine or a pharmaceutically acceptable salt thereof; about 0.5% to about 5% by weight of polyvinylpyrrolidone; about 5% to about 40% by weight of hydroxypropyl methylcellulose; about 2% to about 20% by weight of microcrystalline cellulose; less than about 3% by weight of silicon dioxide; and less than about 3% by weight of magnesium stearate. In particular embodiments of the preceding aspects, the sustained release formulation includes about 50% by weight of L-arginine monohydrochloride, where the L-arginine is L-arginine monohydrochloride; between about 3% and about 4% by weight of polyvinylpyrrolidone; about 35% by weight of hydroxypropyl methylcellulose; about 10% by weight of microcrystalline cellulose; less than about 1% by weight of colloidal silicon dioxide, where the silicon dioxide is colloidal silicon dioxide; and less than about 1% by weight of magnesium stearate.

In another aspect, the invention provides methods for lowering C-reactive protein in a subject including administering to a subject HMG-CoA reductase inhibitor and a sustained release formulation of L-arginine. The method lowers C-reactive protein in a subject to a greater extent than merely administering HMG-CoA reductase inhibitor alone, or L-arginine alone.

Other features and advantages of the invention will be apparent from the following detailed description and claims.

### BRIEF DESCRIPTION OF THE DRAWINGS

FIG. 1 is a graph depicting the release pattern of a formulation comprising L-arginine and simvastatin.

FIG. 2 is photograph of NMR images of infarct size in a mouse brain treated with L-arginine and simvastatin versus in an untreated mouse brain.

FIG. 3 is a bar graph depicting infarct volume in mice treated with L-arginine, simvastatin and both L-arginine and simvastatin.

FIG. 4 is a bar graph depicting total infarct volume in mice treated with L-arginine and various levels of simvastatin.

FIG. 5 is a flow chart depicting a method of manufacture of sustained release L-arginine tablets.

FIG. 6 is a flow chart depicting a method of manufacture of sustained release L-arginine tablets.

FIG. 7 is a bar graph comparing the performance of sustained release L-arginine formulations.

FIG. 8 is a chart comparing the affect of administration of simvastatin with and without a sustained release L-arginine composition of the present invention on endothelium-dependent vasodilation in humans.

FIG. 9 is a chart summarizing the synergistic effect of administration of simvastatin and a sustained release L-arginine composition of the invention on cholesterol levels in humans.

FIG. 10 is a bar graph demonstrating the effect of simvastatin on cultured human aortic endothelial cells (HAEC) versus untreated cultured HAEC.

### DETAILED DESCRIPTION OF THE INVENTION

The present invention provides methods for the treatment and prevention of vascular diseases and disorders including, but not limited to, cardiovascular, cerebrovascular and peripheral vascular diseases and disorders. The present invention is based, at least in part, on the discovery that the coadministration of an HMG-CoA reductase inhibitor and a sustained release formulation of L-arginine has a surprising synergistic effect in the treatment and prevention of vascular diseases and disorders (including cerebrovascular, cardiovascular and peripheral vascular diseases or disorders), and, in particular, in lowering cholesterol. Moreover, the sustained release L-arginine and, optionally, the HMG-CoA reductase inhibitor, may be used to increase vasodilation, increase NO production, and lower C-reactive protein. In another embodiment, the formulations and methods described herein may be used to delay the onset of the disease, disorder and/or event in, for example, populations at risk for development of vascular diseases or disorders and/or an occurrence of an event. The HMG-CoA reductase inhibitor and the sustained release formulation of L-arginine may be administered to the subject either sequentially or concurrently. The reductase inhibitor and the L-arginine may be contained within a single formulation.

Moreover, the invention provides a sustained release formulation of L-arginine and methods of manufacture that render a composition with an optimal release profile. Furthermore, the formulation and methods of manufacture render a composition that is conveniently compressible, but not excessively friable.

In one embodiment, the formulations used in the methods of the invention comprise at least one sustained release agent (for purposes of the present invention, controlled release and sustained release may be used interchangeably), for example, at least one sustained release agonist of endothelial nitric oxide synthase (e.g., an HMG-CoA reductase inhibitor and/or a precursor of nitric oxide such as L-arginine). In another embodiment, the L-arginine is slowly released into the system of a subject. The slow release of L-arginine creates a pharmacokinetic profile of L-arginine within the plasma that provides NOS with a substantially constant supply of L-arginine needed for the production of NO. The formulations can, therefore, slowly dissolve in vivo and release a substantially uniform amount of L-arginine over a time period to be therapeutically effective for a subject. In another embodiment, the HMG-CoA reductase inhibitor is slowly released into the system of the subject. In a further embodiment, the production of NO is substantially uniform over a prolonged period of time.

In another aspect of the present invention, a composition for the treatment of vascular diseases (including, but not limited to, cardiovascular, cerebrovascular, peripheral vascular diseases and disorders), intermittent claudication, critical limb ischemia, and Alzheimer's Disease is provided in the form of food. Such compositions in the form of food may also be used to increase vasodilation, increase NO production and lower cholesterol. Preferably, the food is in the form of a bar such as a prescription health bar. Use of food enables the provision of larger amounts of L-arginine than could be incorporated into a single tablet. The present invention provides a bar that can provide more than 1 gram of L-arginine as well as other agents, as desired. In one embodiment, the L-arginine is added as an immediate release formulation, e.g., immediate release granulars of L-arginine, to a food bar. In another embodiment, the bar includes a sustained release formulation that includes, e.g., sustained release granulars of L-arginine. In another embodiment, the bar further contains additional agents, such as an HMG-CoA reductase inhibitor. Preferably, the HMG-CoA reductase inhibitor, is a statin such as simvastatin.

### DEFINITIONS

Before further description of the invention, certain terms employed in the specification, examples and claims are, for convenience, collected here.

As used herein, unless otherwise specified, the term “subject” includes mammals. The term “mammals” includes, but is not limited to, dogs, cats, cattle, horses, pigs, and humans.

As used herein, the terms “treat”, “treating”, “treatment” and the like refer to the application or administration of a therapeutic agent or formulation to a patient, or application or administration of a therapeutic agent or formulation to an isolated tissue from a patient, who has a disease or disorder, a symptom of disease or disorder or a predisposition toward a disease or disorder, with the purpose of curing, healing, alleviating, relieving, altering, remedying, preventing, ameliorating, delaying onset of the disease or disorder and/or event, slowing the progression of the disease or disorder, improving or affecting the disease or disorder, the symptoms of disease or disorder or the predisposition toward a disease or disorder and/or event.

As used herein, the term “vascular disease” or “vascular disorder” generally refer to diseases or disorders of blood vessels and include, but are not limited to, cardiovascular, cerebrovascular, and peripheral vascular diseases or disorders. Cardiovascular disease refers to diseases of blood vessels of the heart. See, e.g., Kaplan, R. M., et al., “Cardiovascular diseases” in Health and Human Behavior, pp. 206-242 (McGraw-Hill, New York 1993). Cardiovascular disease is generally one of several forms, including, for example, hypertension (also referred to as high blood pressure), coronary heart disease, stroke, and rheumatic heart disease. Peripheral vascular disease or disorders refer to diseases of any of the blood vessels outside of the heart. For example, peripheral vascular disease may refer to a narrowing of the blood vessels that carry blood to leg and arm muscles. Cerebrovascular disease refers to diseases that affect the ability of blood vessels to supply blood to the brain.

The term “atherosclerosis” encompasses vascular diseases and disorders and conditions that are recognized and understood by physicians practicing in the relevant fields of medicine. Atherosclerotic cardiovascular disease, coronary heart disease (also known as coronary artery disease or ischemic heart disease), cerebrovascular disease and peripheral vessel disease are all clinical manifestations of atherosclerosis and are therefore encompassed by the terms “atherosclerosis” and “atherosclerotic disease”.

As used herein the terms “coadministration” or “coadministered” when used to describe the administration of two or more compounds to a subject means that the compounds, which may be administered by the same or different routes, are administered concurrently (e.g., as a mixture) or sequentially, such that the pharmacological effects of each overlap in time. As used herein, unless otherwise specified, when applied to the administration of at least two compounds, the term “sequentially” means that the compounds are administered such that the pharmacological effects of each overlap in time. In certain embodiments, agents are coadministered substantially simultaneously. By “substantially simultaneously,” it is meant that the formulation of the invention is administered to the subject close enough in time with the administration of at least one additional agent, whereby the agents may exert an additive or even synergistic effect, e.g., without limitation, increasing NOS activity, NO production, or vasodilation.

As used herein the term “precursor of NO” includes any substrate precursor of native NO, e.g., L-arginine.

The term “native NO” as used herein refers to nitric oxide that is produced through the bio-transformation of L-arginine or the L-arginine dependent pathway. The terms “endothelium derived relaxing factor (EDRF)” or “endothelium derived nitric oxide (EDNO)” may be used interchangeably with “native NO”.

As used herein the term “L-arginine” refers to L-arginine and all of its biochemical equivalents, e.g., L-arginine hydrochloride, precursors, and its basic form, that act as substrates of NOS with resulting increase in production of NO. The term includes pharmaceutically acceptable salts of L-arginine.

The term “pharmaceutically acceptable salts” refers to salts prepared from pharmaceutically acceptable non-toxic acids or bases including inorganic acids and bases and organic acids and bases. Suitable non-toxic acids include inorganic and organic acids such as acetic, benzenesulfonic, benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. Particularly preferred are hydrochloric, hydrobromic, phosphoric, and sulfuric acids, and most particularly preferred is the hydrochloride salt.

Since the L-arginine used in the methods of the present invention is both basic and acidic, salts may be prepared from pharmaceutically acceptable non-toxic acids or bases including inorganic and organic acids or inorganic and organic bases. Such salts may contain any of the following anions: acetate, benzensulfonate, benzoate, camphorsulfonate, citrate, fumarate, gluconate, hydrobromide, hydrochloride, lactate, maleate, mandelate, mucate, nitrate, pamoate, phosphate, succinate, sulfate, tartrate, and the like. Particularly preferred are benzensulfonate, hydrobromate, hydrochloride, and sulfate. Such salts may also contain the following cations: aluminum, calcium, lithium, magnesium, potassium, sodium, zinc, benzathine, chloroprocaine, choline, diethanolamine, ethylenediamine, meglumine, and procaine.

As used herein the term “agonist” or “agonist of eNOS or cNOS” refers to an agent which stimulates the bio-transfornation of a substrate such as, for example, L-arginine to NO. An agonist of eNOS or cNOS includes, for example, an HMG-CoA reductase inhibitor. “HMG-CoA reductase (3-hydroxy-3-methylglutaryl-coenzyme A)” is the microsomal enzyme that catalyzes the rate limiting reaction in cholesterol biosynthesis. An “HMG-CoA reductase inhibitor” inhibits HMG-CoA reductase. HMG-CoA reductase inhibitors are also referred to as “statins.”

There are a large number of compounds described in the art that have been obtained naturally or synthetically, which inhibit HMG-CoA reductase and are referred to as “statins,” and which form the category of agents useful for practicing the present invention. Examples include, without limitation, those which are commercially available, such as simvastatin (U.S. Pat. No. 4,444,784), lovastatin (U.S. Pat. No. 4,231,938), pravastatin sodium (U.S. Pat. No. 4,346,227), fluvastatin (U.S. Pat. No. 4,739,073), atorvastatin (U.S. Pat. No. 5,273,995), cerivastatin, rosuvastatin, and numerous others such as compactin, dalvastatin, mevastatin, fluindostatin, pitavastatin, HR-780, GR-95030, CI 980, BMY 22089, BMY 22566, and those described in, for example, U.S. Pat. No. 5,622,985, U.S. Pat. No. 5,135,935, U.S. Pat. No. 5,356,896, U.S. Pat. No. 4,920,109, U.S. Pat. No. 5,286,895, U.S. Pat. No. 5,262,435, U.S. Pat. No. 5,260,332, U.S. Pat. No. 5,317,031, U.S. Pat. No. 5,283,256, U.S. Pat. No. 5,256,689, U.S. Pat. No. 5,182,298, U.S. Pat. No. 5,369,125, U.S. Pat. No. 5,302,604, U.S. Pat. No. 5,166,171, U.S. Pat. No. 5,202,327, U.S. Pat. No. 5,276,021, U.S. Pat. No. 5,196,440, U.S. Pat. No. 5,091,386, U.S. Pat. No. 5,091,378, U.S. Pat. No. 4,904,646, U.S. Pat. No. 5,385,932, U.S. Pat. No. 5,250,435, U.S. Pat. No. 5,132,312, U.S. Pat. No. 5,130,306, U.S. Pat. No. 5,116,870, U.S. Pat. No. 5,112,857, U.S. Pat. No. 5,102,911, U.S. Pat. No. 5,098,93 1, U.S. Pat. No. 5,081,136, U.S. Pat. No. 5,025,000, U.S. Pat. No. 5,021,453, U.S. Pat. No. 5,017,716, U.S. Pat. No. 5,001,144, U.S. Pat. No. 5,001,128, U.S. Pat. No. 4,997,837, U.S. Pat. No. 4,996,234, U.S. Pat. No. 4,994,494, U.S. Pat. No. 4,992,429, U.S. Pat. No. 4,970,231, U.S. Pat. No. 4,968,693, U.S. Pat. No. 4,963,538, U.S. Pat. No. 4,957,940, U.S. Pat. No. 4,950,675, U.S. Pat. No. 4,946,864, U.S. Pat. No. 4,946,860, U.S. Pat. No. 4,940,800, U.S. Pat. No. 4,940,727, U.S. Pat. No. 4,939,143, U.S. Pat. No. 4,929,620, U.S. Pat. No. 4,923,861, U.S. Pat. No. 4,906,657, U.S. Pat. No. 4,906,624 and U.S. Pat. No. 4,897,402, the disclosures of each of which are incorporated herein by reference. Any other member of the class of compounds that inhibits HMG-CoA reductase may be used in the methods of the invention. A combination of two or more HMG-CoA reductase inhibitors may also be used in the methods of the invention.

The term “eNOS activity”, as used herein, means the ability of a cell to generate NO from the substrate L-arginine. Increased eNOS activity can be accomplished in a number of different ways. For example, an increase in the amount of eNOS protein or an increase in the activity of the protein (while maintaining a constant level of the protein) can result in increased “activity.” An increase in the amount of protein available can result from, for example and without limitation, increased transcription of the eNOS gene, increased translation of eNOS mRNA, increased stability of the eNOS MRNA, activation of eNOS, or a decrease in eNOS protein degradation.

The eNOS activity in a cell or in a tissue can be measured in a variety of different ways. A direct measure is to measure the amount of eNOS present. Another direct measure is to measure the amount of conversion of L-arginine to L-citrulline by eNOS or the amount of nitric oxide generation by eNOS under particular conditions, such as the physiologic conditions of the tissue. The eNOS activity also can be measured indirectly, for example by measuring mRNA half-life (an upstream indicator) or by a phenotypic response to the presence of NO (a downstream indicator). One phenotypic measurement employed in the art is measuring endothelial dependent relaxation in response to acetylcholine, which response is affected by eNOS activity. The level of NO present in a sample can be measured using a NO meter. All of the foregoing techniques are well known to those of ordinary skill in the art.

The methods of the present invention, by causing an increase in NO production, permit not only the re-establishment of normal base-line levels of eNOS activity, but also allow increasing such activity above normal base-line levels. Normal base-line levels are the amounts of activity in a normal control group, controlled for age and having no symptoms that would indicate alteration of endothelial cell NOS activity (such as hypoxic conditions, hyperlipidemia and the like). The actual level then will depend upon the particular age group selected and the particular measure employed to assess activity. In abnormal circumstances, endothelial cell NOS activity (and NO production) is depressed below normal levels. Accordingly, the formulations of the invention can not only restore normal base-line levels of NO production in such abnormal conditions, but can increase endothelial cell NOS activity (and NO production) far above normal base-line levels.

The term “carrier” refers to diluents, excipients and the like for use in preparing admixtures of a pharmaceutical composition.

As used herein, the term “dosage form” means a pharmaceutical composition that contains an appropriate amount of active ingredient for administration to a subject, e.g., a patient either in single or multiple doses.

The unit “mg/Kg” as used herein means the mg of agent per Kg of subject body weight.

As used herein, unless otherwise indicated, the term “half-life” means the time taken to decrease the concentration of drug in the blood plasma of the organism by about one half from the drug concentration at the time of administration.

As used herein, unless otherwise specified, the term “immediate release” means that no extrinsic factors delay the in vitro release of one or more drugs.

As used herein, the terms “pharmaceutical composition” or “pharmaceutical formulation,” used interchangeably herein, mean a composition that comprises pharmaceutically acceptable constituents.

As used herein, the term “pharmaceutically acceptable” means the type of formulation that would be reviewed and possibly approved by a regulatory agency of the Federal or a state government or listed in the U.S. Pharmacopeia or other generally recognized pharmacopeia for use in animals, and more particularly in humans.

As used herein unless otherwise specified, the term “pharmaceutically acceptable carrier” means a carrier medium which does not interfere with the effectiveness of the biological activity of the active ingredient and which is not toxic to the subject to which it is administered. The use of such media and agents for pharmaceutically active formulations is well known in the art. Except insofar as any conventional media or agent is incompatible with the active compound, use thereof in the formulations used in the methods of the invention is contemplated.

As used herein, the term “pharmaceutically acceptable salts” refers to salts prepared from pharmaceutically acceptable non-toxic acids, including inorganic acids and organic acids.

As used herein unless otherwise specified, the term “pharmaceutically acceptable carrier” means a carrier medium which does not interfere with the effectiveness of the biological activity of the active ingredient and which is not toxic to the subject to which it is administered. The use of such media and agents for pharmaceutically active formulations is well known in the art. Except insofar as any conventional media or agent is incompatible with the active compound, use thereof in the formulations used in the methods of the invention is contemplated.

As used herein, the term “pharmaceutically acceptable salts” refers to salts prepared from pharmaceutically acceptable non-toxic acids, including inorganic acids and organic acids.

As used herein, unless otherwise specified, the term “sustained release” is defined as a prolonged release pattern of one or more drugs, such that the drugs are released over a period of time. A sustained release formation is a formulation with a release kinetics which results in measurable serum levels of the drug over a period longer than that obtained following IV injection or by administering an immediate release oral dosage form. A sustained release formulation is giving a continued effect to drugs of which biological half lives after administration are short; decreasing side effects of drugs which likely exhibit side effect Cₘₐₓ-dependently; and improving compliance by decreasing the number of times of administration. For purposes of the present invention, sustained release, slow release, controlled release, extended release, prolonged release, controlled release and delayed release are used interchangeably.

As used herein, the term “salt or complex” is used to describe a compound or composition comprising two or more chemical moieties that are associated by at least one type of interaction including, but not limited to, Van der Waals, ionic and/or hydrogen bonding. A salt or complex may exist as a solid or in a liquid.

As used herein, the term “weight percent” when used to describe the amount of a component within a formulation means the weight of the specified component based upon the weight of all components within the formulation.

Various aspects of the invention are described in further detail in the following subsections:

I. Formulations Used In Methods of Treatment or Prevention of Cerebrovascular and Cardiovascular Diseases and Disorders

The methods of the invention include methods of treating and preventing cerebrovascular and/or cardiovascular diseases or disorders in a subject, e.g., a human, comprising administering to the subject a formulation comprising an HMG-CoA reductase inhibitor and a formulation comprising L-arginine, either concurrently or sequentially. Alternatively, a single formulation comprising L-arginine and an HMG-CoA reductase inhibitor is administered to a subject.

One embodiment of the invention encompasses formulations comprising L-arginine in a sustained release formulation, an HMG-CoA reductase inhibitor in a sustained release formulation, or both L-arginine and an HMG-CoA reductase inhibitor in a sustained release formulation. In one embodiment, the invention encompasses formulations comprising L-arginine that may be administered either concurrently or sequentially with at least one HMG-CoA reductase inhibitor wherein the formulation releases L-arginine in a substantially constant concentration over a prolonged period of time and the HMG-CoA reductase inhibitor is present in an immediate release formulation. In another embodiment, the invention encompasses formulations comprising L-arginine in a high concentration and in a sustained release formulation wherein the pharmacokinetic profile is zero order release kinetics (i.e., linear release rate over time). The release characteristics of both classes of drugs may be modified to provide release patterns that allow for the adaptation of the combination into a once daily single unit dosage.

In one embodiment, the formulations used in the methods of the invention comprise L-arginine in a therapeutically effective amount, an HMG-CoA reductase inhibitor in a therapeutically effective amount, and at least one sustained release agent. The formulations also can include additional ingredients necessary to modify the formulations for administration, preservation, esthetics and the like. In one embodiment, the formulation of the present invention also include binders, fillers and lubricants. In a preferred embodiment, the formulation comprises a sustained release L-arginine formula comprising L-arginine, a binder, one or more sustained release agents, a glidant, and a release agent or lubricant. The formulation may further comprise fillers and/or compression agents. The sustained release formulations of the present invention are particularly advantageous because their release profile allows the administration of lower dosages to maintain the same level of drug in the body than required with immediate release or commercially available sustained release agents. Because administration of the sustained release L-arginine with a statin can also increase the effectiveness of the statin, e.g., simvastatin, the use of the formulations of the invention may also allow a lower dosage of statin with an equivalent beneficial affect.

L-arginine is commercially available from a number of sources known to the skilled practitioner. USP grade L-arginine, for example, is commercially available from various sources including Sigma-Aldrich (Milwaukee, Wis.). Suitable arginine and arginine derivative compounds include, but are not limited to, arginine salts such as arginine HCl, arginine aspartate, or arginine nicotinate. Other arginine compounds or derivatives may be chosen from di-peptides that include arginine such as alanylarginine (ALA-ARG), valinyL-arginine (VAL-ARG), isoleucinyL-arginine (ISO-ARG), and leucinyL-arginine (LEU-ARG), and tri-peptides that include arginine such as argininyl-lysinyl-glutamic acid (ARG-LYS-GLU) and arginyl-glysyL-arginine (ARG-GLY-ARG). The L-arginine preferably is L-arginine monohydrochloride.

In one embodiment, the L-arginine is present at about 10% to about 75% by weight of the formulation. In another embodiment, the L-arginine is present at about 25% to about 75% by weight of the formulation. In a preferred embodiment, the L-arginine is present at about 50% by weight of the formulation.

Use of one or more sustained release agents allows for the slow release of the L-arginine and/or the HMG-CoA reductase inhibitor over an extended period of time. For example, the sustained release agent may release L-arginine at a rate that will not cause concentration peaks or lows that would exacerbate side effects associated with high or low concentrations of L-arginine within the bloodstream. Sustained release agents suitable for the formulations used in the methods of the present invention include hydration agents, e.g., such as cellulose, that partially hydrate when in contact with an aqueous envirorunent to form a gelatinous barrier that retards dissolution of the agent that the hydration agent is coating. In other words, the sustained release agents form a temporary barrier to water such that water is slowly absorbed into the formulation thereby hydrating the formulation and subsequently releasing the active ingredient, e.g., L-arginine, at a rate substantially slower than a formulation without sustained release agents. Additionally, the sustained release agents are present in a particle size where upon incorporation into a capsule or compaction or compression into a tablet, pill, or gelcap water slowly permeates into the structure.

In one embodiment, the sustained release agent or agents include, but are not limited to, cellulose ether products, polymethylmethacrylate, or polyvinylalcohol. In another embodiment, sustained release agents include celluloses including, but not limited to methylcellulose, hydroxypropyl methylcellulose, hydroxyethylcellulose, or combinations thereof. In a preferred embodiment, the sustained release agents include one or more hydroxypropyl methylcelluloses. Suitable sustained release agents are commercially available from The Dow Chemical Company under the trade designations METHOCEL® and ETHOCEL®. In a preferred embodiment, the sustained release agent is METHOCEL® K100 M CR Premium and/or METHOCEL® E 4M CR Premium.

The sustained release agent is typically present in an amount sufficient to release the active ingredient, e.g., L-arginine or an HMG-CoA reductase inhibitor, over a desired period of time. In one embodiment, the sustained release agent is present in an amount of about 5% to about 40% by weight of the formulation. In another embodiment, the sustained release agent is present in an amount of about 5% to about 75% by weight. In yet another embodiment, the sustained release agent is present in an amount of about 15% to about 50% by weight of the formulation. In a preferred embodiment, the sustained release agent(s) is present at about 35% by weight of the formulation. All ranges within each of the above ranges are within the scope of the present invention.

In certain embodiments, the formulation may contain less than about 7 g L-arginine, for example, less than about 6 g, about 5 g, about 4 g, about 3 g, about 2 g, or about 1 g L-arginine. For example, the formulation may contain from about 1 g to about 7 g, about 2 g to about 6 g or about 3 g to about 5 g L-arginine. For example, ranges of values using a combination of any of the above recited values as upper and/or lower limits are intended to be included. Preferably, the formulation contains less than about 4 g L-arginine. While not risking to be bound by theory, the sustained release formulations of L-arginine allow for a small dosage to be employed, i.e. the total amount of L-arginine may be lower and yet still achieve a therapeutic effect.

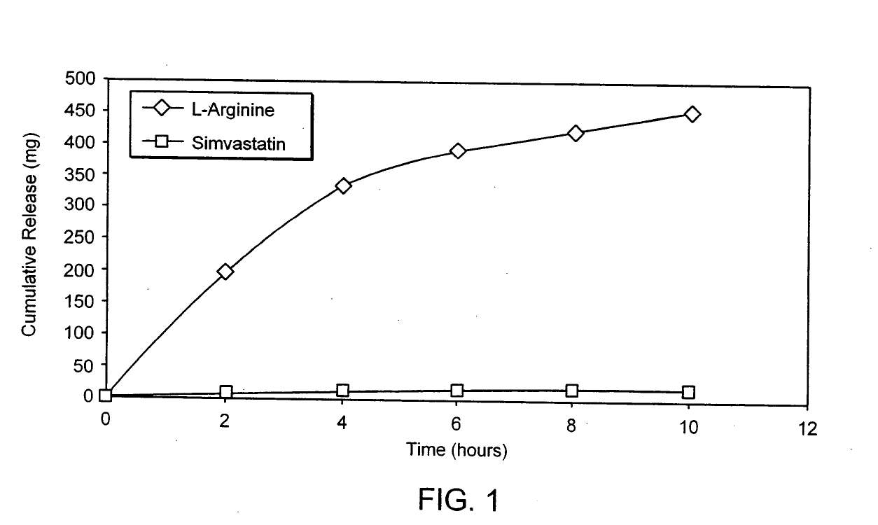

In one embodiment, the sustained release agent releases L-arginine over a period of 10 hours, as depicted in FIG. 1. In one embodiment, the formulation releases L-arginine substantially uniformly over a period from about 4 hours to about 24 hours. In another embodiment, the formulation of the present invention releases L-arginine substantially uniformly over a period of about 8 hours to about 24 hours. In yet another embodiment, the sustained release L-arginine formulation releases L-arginine substantially uniformly over a period of about 12 hours to about 48 hours.

In another embodiment, a formulation used in the methods of the present invention will release L-arginine in a manner to provide a pharmacokinetic profile wherein the half-life (T_{1/2}) and the Tₘₐₓ are sufficient to maintain L-arginine at a substantially constant level. In other words, in one embodiment, a sustained release formulation of the invention releases L-arginine such that a steady state of circulating L-arginine is achieved and remains constant. In one embodiment, the pharmacokinetic profile is such that T_{1/2} is from about 4 hours to about 12 hours and the Tₘₐₓ is about 4 hours. In yet another embodiment, T_{1/2} is from about 4 hours to about 8 hours and the Tₘₐₓ is about 4 hours.

Binders useful in the formulation include those commonly known to the skilled practitioner. Binders include, but are not limited to, sugars, such as lactose, sucrose, glucose, dextrose, and molasses; natural and synthetic gums, such as acacia, guar gum, sodium alginate, extract of Irish moss, panwar gum, ghatti gum; other binders include a mixture of polyethylene oxide and polyethylene glycol, methylcellulose, sodium carboxymethylcellulose, hydroxypropyl cellulose (HPC), hydroxyethyl cellulose, hydroxypropyl methylcellulose, alginic acid, ethyl cellulose, microcrystalline cellulose, carbomer, zein, starch, dextrin, maltodextrin, gelatin, pregelatinized starch, polyvinlypyrrolidone (PVP) or povidone, and mixtures thereof. In a preferred embodiment, the binder is polyvinylpyrrolidone homopolymer.

In one embodiment, the binder is present at less than about 20% by weight of the formulation. In another embodiment, the binder is present at about 0.5% to about 5% by weight of the formulation. In a preferred embodiment, the binder is present at about 3% to about 4% by weight of the formulation.

In a preferred embodiment, the formulation of sustained release L-arginine also includes a glidant. The glidant can be any known USP grade glidant including, e.g., silicon dioxide. In a preferred embodiment, the glidant is colloidal silicone dioxide.

In one embodiment, the glidant is present at less than about 3% by weight of the formulation. In another embodiment, the glidant is present at less than about 2% of the formulation. In a preferred embodiment, the glidant is present at less than about 1% by weight of the formulation.

Fillers useful in the formulation include those commonly known to the skilled artisan. Typical fillers include, but are not limited to, sugars such as lactose, sucrose, dextrose, mannitol, and sorbitol, whey, dibasic calcium phosphate, tribasic calcium phosphate, calcium sulfate, and mixtures thereof. Other fillers include, but are not limited to, cellulose preparations such as maize starch, wheat starch, rice starch, potato starch, gelatin, gum tragacanth, methyl cellulose, hydroxypropyl methylcellulose, sodium carboxymethylcellulose, polyvinylpyrrolidone, and mixtures thereof. Microcrystalline cellulose can also function as a compression agent as well as a filler. In a preferred embodiment the filler/compression agent is microcrystalline cellulose. More preferably, the microcrystalline cellulose is that sold under the designation AVICEL® PH 102 by The Dow Chemical Company.

In one embodiment, the filler is present at less than about 50% by weight of the formulation. In another embodiment, the filler is present at about 2% to about 20% by weight of the formulation. In a preferred embodiment, the filler is present at about 10% by weight of the formulation.

Excipients can be added to increase the amount of solids present in the formulation. Among the excipients found useful for this purpose, often in combination, are sodium or potassium phosphates, calcium carbonate, calcium phosphate, sodium chloride, citric acid, tartaric acid, gelatin, and carbohydrates such as dextrose, sucrose, lactose, sorbitol, inositol, mannitol and dextran, starches, cellulose derivatives, gelatin, and polymers such as polyethylene glycols. In addition to those mentioned herein, others are known to those skilled in the art.

Release agents or lubricants useful in the formulation include those commonly known to the skilled artisan. Typical lubricants include, but are not limited to, stearate, magnesium stearate, zinc stearate, calcium stearate, stearic acid, hydrogenated vegetable oils (e.g., hydrogenated cottonseed oil), sodium stearyl fumarate, glyceryl palmitostearate, glyceryl behenate, sodium benzoate, sodium lauryl sulfate, magnesium lauryl sulfate, mineral oil, talc, and mixtures thereof. In a preferred embodiment, the lubricant is magnesium stearate. In other embodiments, lubricants are chosen so as to insure optimal absorption and utilization of nutrients.

In one embodiment, the lubricant is present at less than about 20% by weight of the formulation. In another embodiment, the lubricant is present at about 2% to about 20% by weight of the formulation. In a preferred embodiment, the lubricant is present at about 10% by weight of the formulation.

Disintegrants include, but are not limited to, sodium starch glycolate, croscarmellose sodium, crospovidone, cross-linked polyvinylpyrrolidone, corn starch, pregelatinized starch, microcrystalline cellulose, alginic acid, amberlite ion exchange resins, polyvinylpyrrolidone, polysaccharides, sodium carboxymethylcellulose, agar, salts thereof such as sodium alginate, Primogel, and mixtures thereof.

The compression agent allows for the formulation to be shaped into a tablet, troche, gelcap, or other presentation for administration in solid form. In one embodiment, the compression agent allows the formulation to be shaped into a tablet, troche, or gelcap. Compression agents include, but are not limited to, Avicel, magnesium stearate, wax, gums, celleusics, stearate, or combinations thereof. In a preferred embodiment, the compression agent is microcrystalline cellulose.

In one embodiment, the compression agent is present in an amount of about 0.01% to about 5% by weight percent of the formulation. In another embodiment, the compression agent is present in an amount of about 0.5% to about 3%. In yet another embodiment, the compression agent is present in an amount of about 1% to about 2% by weight of the formulation.

In one embodiment, the L-arginine formula includes L-arginine in a unit dosage that would be sufficient for about 5 mg/Kg to about 40 mg/Kg subject body weight. In another embodiment, the L-arginine formula includes L-arginine in a unit dosage that would be sufficient for about 20 mg/Kg to about 25 mg/Kg.

In another embodiment, both L-arginine and an HMG-CoA reductase inhibitor are in a sustained release formulation. The amount of HMG-CoA reductase inhibitor may vary based on the specific inhibitor present in the formulation, as some inhibitors are more efficacious than others. For example, BAYCOL® may be present in an amount of about 0.1 mg to about 0.8 mg per tablet, and ZOCOR® may be present in an amount of about 10 mg to about 80 mg per tablet. Those skilled in the art will be able to determine a therapeutic amount based on the specific inhibitor employed. In one embodiment, the HMG-CoA reductase inhibitor is simvastatin and is present in a unit dosage that would be sufficient for about 0.5 mg/Kg to about 3 mg/Kg subject body weight. In another embodiment, the HMG-CoA reductase inhibitor is simvastatin and is present in a unit dosage that would be sufficient for about 1.2 mg/Kg to about 1.4 mg/Kg subject body weight.

In yet another embodiment, the L-arginine and HMG-CoA reductase inhibitor are both provided in separate sustained release formulations, e.g., separate tablets. Sustained release HMG-CoA reductase inhibitor is commercially available from, e.g., Merck & Company, Inc. (Rahway, N.J.).

Formulations used in the methods of the invention may comprise a pharmaceutical carrier according to conventional pharmaceutical compounding techniques. The carrier may take a wide variety of forms depending on the form of the preparation desired for oral administration. In preparing the formulations for oral dosage form any of the usual pharmaceutical media may be employed. The most preferred oral solid preparations are tablets and gelcaps. Alternatively, the formulations of the present invention may be incorporated into a capsule. In this embodiment, the sustained release L-arginine granulars, and, optionally, the HMG-CoA reductase inhibitor, may be incorporated within a capsule.

Because of their ease of administration, tablets and capsules represent the most advantageous oral dosage unit form, in which case solid pharmaceutical carriers are employed. Tablets or capsules may contain an L-arginine formulation and HMG-CoA reductase inhibitor formulation in the same tablet or capsule in different configurations. Configurations include, a two-part half and half tablet or capsule, one formulation surrounding a second, dispersion of one formulation in another, granules of both formulations intermixed, and the like. If desired, tablets or capsules may be coated by standard aqueous or non-aqueous techniques.

The formulations used in the methods of the present invention may also comprise other pharmaceutically acceptable ingredients, such as those commonly used in the art. See, Remington: the Science & Practice of Pharmacy, by Alfonso R. Gennaro, 20th ed., Williams & Wilkins, 2000. Additional ingredients used in the formulations used in the methods of the present invention include, but are not limited to, water, glycols, oils, alcohols, starches, sugars, diluents, disintegrating agents, preservatives, excipients, lubricants, disintegrants, diluents, carriers, stabilizing agents, coloring agents, flavoring agents, and combinations thereof. Examples of suitable diluents include water, ethanol, polyols, vegetable oils, injectable organic esters such as ethyl oleate, and combinations thereof. Formulations can also contain adjuvants such as preserving, wetting, emulsifying, and dispensing agents. Prevention of the action of microorganisms can be insured by various antibacterial and antifungal agents including, but not limited to, parabens, chlorobutanol, phenol, sorbic acid, and the like. It may also be desirable to include isotonic agents including, but not limited to, sugars, sodium chloride, and the like.

In another embodiment of the invention, the formulations may be further co-administered with at least one other pharmaceutical agent. Examples of categories of pharmaceutical agents include: adrenergic agent; adrenocortical steroid; adrenocortical suppressant; aldosterone antagonist; amino acid; ammonia detoxicant; anabolic; analeptic; analgesic; androgen; anesthetic; anorectic; antagonist; anterior pituitary suppressant; anthelmintic; anti-acne agent; anti-adrenergic; anti-allergic; anti-amebic; anti-androgen; anti-anemic; anti-anginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiuretic; anti-emetic; anti-epileptic; anti-estrogen; antifibrinolytic; antifungal; antiglaucoma agent; antihemophilic; antihemorrhagic; antihistamine; antihyperlipidemia; antihyperlipoproteinemic; antihypertensive; anti-infective; anti-inflammatory; antikeratinizing agent; antimalarial; antimicrobial; antimigraine; antimitotic; antimycotic, antinauseant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinesterase deactivator; coccidiostat; cognition adjuvant; depressant; diuretic; dopaminergic agent; ectoparasiticide; emetic; enzyme inhibitor; estrogen; fibrinolytic; fluorescent agent; free oxygen radical scavenger; gastrointestinal motility effector; glucocorticoid; gonad-stimulating principle; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; impotence therapy adjunct; keratolytic; LNRII agonist; liver disorder treatment; luteolysin; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; potentiator; progestin; prostaglandin; prostate growth inhibitor; prothyrotropin; psychotropic; radioactive agent; regulator; relaxant; repartitioning agent; scabicide; sclerosing agent; sedative; selective adenosine A1 antagonist; serotonin antagonist; serotonin inhibitor; serotonin receptor antagonist; steroid; stimulant; suppressant; symptomatic multiple sclerosis; synergist; thyroid hormone; thyroid inhibitor; thyromimetic; tranquilizer; treatment of cerebral ischemia; treatment of Paget's disease; treatment of unstable angina; uricosuric; vasoconstrictor; vasodilator; vulnerary; wound healing agent; or xanthine oxidase inhibitor.

Another example of a pharmaceutical agent includes angiotensin converting enzyme inhibitors (ACE inhibitors). ACE is an enzyme that catalyzes the conversion of angiotensin I to angiotensin II. ACE inhibitors include amino acids and derivatives thereof, peptides, including di and tri peptides and antibodies to ACE which intervene in the renin-angiotensin system by inhibiting the activity of ACE thereby reducing or eliminating the formation of pressor substance angiotensin II. ACE inhibitors have been used medically to treat hypertension, congestive heart failure, myocardial infarction and renal disease. Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril. Estrogens upregulate NOS expression whereas ACE inhibitors do not affect expression, but instead influence the efficiency of the action of NOS on L-arginine. Thus, activity can be increased in a variety of ways. In general, activity is increased by the reductase inhibitors of the invention by increasing the amount of the active enzyme present in a cell versus the amount present in a cell absent treatment with the reductase inhibitors according to the invention.

II. Prophylactic and Therapeutic Methods

In one aspect, the invention provides methods for preventing vascular diseases or disorders, such as cerebrovascular and/or cardiovascular diseases or disorders, in a subject by administering to a subject at risk for cerebrovascular and/or cardiovascular diseases or disorders a formulation comprising L-arginine along with a formulation comprising an HMG-CoA reductase inhibitor (e.g., simvastatin), either sequentially, or concurrently, or a single formulation comprising L-arginine along with an HMG-CoA reductase inhibitor. Subjects at risk for cerebrovascular and/or cardiovascular diseases and disorders (including events) can be identified by, for example, a predisposition to atherosclerosis, symptoms of atherosclerosis, or by the presence of risk factors such as, for example, cigarette smoking, high blood pressure, diabetes, family history, genetic factors, high cholesterol levels, advancing age and alcohol use.

Administration of a formulation used in the methods of the invention as a prophylactic agent can occur prior to the manifestation of symptoms characteristic of the onset of cerebrovascular and/or cardiovascular disease or disorder, such that cerebrovascular and/or cardiovascular disease or disorder is prevented, its progression slowed, or its onset delayed.

As described in International Patent Publication No. WO 00/56403 entitled “Upregulation of Type III Endothelial Cell Nitric Oxide Synthase By HMG-CoA Reductase Inhibitors,” incorporated in its entirety by this reference, upregulation of NOS activity does not depend upon a decrease in cholesterol synthesis and in particular does not depend upon a decrease in the formation of ox-LDL. The present invention, therefore, is useful whenever it is desirable to restore eNOS activity or increase such activity in an affected cell or tissue. The tissue is defined as to include both the cells in the vasculature supplying nutrients to the tissue, as well as cells of the tissue that express eNOS.

Nitric Oxide Synthase activity is involved in many conditions, including impotence, heart failure, gastric and esophageal motility disorders, kidney disorders such as kidney hypertension and progressive renal disease, insulin deficiency, etc. Individuals with such conditions may benefit from increased NO production. For example, individuals with pulmonary hypertension often have reduced levels of Nitric Oxide Synthase expression in their pulmonary vessels and benefit clinically from inhalation of Nitric Oxide. The invention therefore is particularly useful for treating pulmonary hypertension. It also has been demonstrated that hypoxia causes an inhibition of eNOS activity. The invention therefore is useful for treating subjects with hypoxia-induced conditions. It also has been discovered, surprisingly, that HMG-CoA reductase inhibitors are useful for reducing ID brain injury that occurs following a stroke.

The subject can have a condition characterized by an abnormally low level of eNOS activity which is hypoxia-induced. In other embodiments, the subject can have a condition comprising an abnormally low level of eNOS activity that is chemically induced. In still other embodiments the subject can have a condition comprising an abnormally low level of eNOS activity that is cytokine induced. In certain important embodiments, the subject has pulmonary hypertension or an abnormally elevated risk of pulmonary hypertension. In other important embodiments, the subject has experienced an ischemic stroke or has an abnormally elevated risk of an ischemic stroke. In still other important embodiments, the subject has heart failure or progressive renal disease. In yet other important embodiments, the subject is chronically exposed to hypoxic conditions.

In further important embodiments, the subject has experienced a thrombotic event or has an abnormally elevated risk of thrombosis. In still other embodiments, the subject has an abnormally elevated risk of arteriosclerosis or has arteriosclerosis. In other important embodiments, the subject has an abnormally elevated risk of developing a myocardial infarction or has experienced a myocardial-infarction. In yet another embodiment, the subject has an abnormally elevated risk of reperfusion injury. In preferred embodiments, the subject with an elevated risk of reperfusion injury is an organ transplant recipient (e.g., heart, kidney, liver, etc.). In other important embodiments, the subject has homocystinuria. In certain other important embodiments, the subject has cerebral autosonial dominant arteriopathy with subcortical infarcts and leucoencephalopathy (CADASIL) syndrome. In further important embodiments, the subject has a degenerative disorder of the nervous system. In preferred embodiments, the subject with a degenerative disorder of the nervous system has Alzheimer's disease.

In certain other embodiments, when the subject in need of a treatment according to the present invention has an abnormally elevated risk of an ischemic stroke, HMG-CoA reductase inhibitors are excluded as treatments for such subjects.

In other embodiments, the methods and compositions (e.g., L-arginine sustained release formulations, L-arginine food bars, etc.) of the present invention may be used to treat or prevent Alzheimer's Disease. In yet another embodiment, the methods and compositions of the present invention may be used to treat or prevent intermittent claudication. In yet another embodiment, the formulations and compositions of the present invention may be used to increase vasodilation.

In a preferred embodiment, the methods of the present invention may be used to lower cholesterol levels in a subject. Administering HMG-CoA reductase inhibitor and L-arginine to a subject can serve to lower total cholesterol. In one embodiment, the method lowers total cholesterol by about 50 to about 150 mg/dL. In another embodiment, the method reduces total cholesterol by about 80 to about 100 mg/dL. In addition, administering HMG-CoA reductase inhibitor and L-arginine to a subject can serve to lower low density lipoprotein (LDL) cholesterol. In one embodiment, the method lowers LDL cholesterol by about 40 to about 110 mg/dL. In another embodiment the method lowers LDL cholesterol by about 60 to about 100 mg/dL. The methods of the present invention may also serve to increase high density lipoprotein (HDL) cholesterol in a subject. Furthermore, the administration of HMG-CoA reductase inhibitor and L-arginine can lower triglycerides in a subject. In one embodiment, the methods of the invention lower triglycerides in a subject by about 30 to about 100 mg/dL. In another embodiment, the methods of the invention lower triglycerides by about 45 to about 75 mg/dL.

The coadministration of HMG-CoA reductase inhibitor and L-arginine has a synergistic effect in reducing cholesterol levels in a subject. The methods and compositions of the present invention have been shown to reduce cholesterol levels at a surprising and significant amount over other known methods and compositions. In particular, the coadministration of HMG-CoA reductase inhibitor and sustained release L-arginine in accordance with the present invention reduces triglycerides and LDL levels in a significant manner over preexisting methods. Moreover, the coadministration of HMG-CoA reductase inhibitor and sustained release L-arginine increase HDL in a significant manner over preexisting methods. In one embodiment, the coadministration of HMG-CoA reductase and L-arginine lowers total cholesterol by about 5% to about 15% more compared to administration of HMG-CoA reductase inhibitor alone. In another embodiment, the coadministration of HMG-CoA reductase and L-arginine lowers total cholesterol by about 5 to about 20 mg/dL more compared to administration of HMG-CoA reductase inhibitor alone. In yet another embodiment, the coadministration of HMG-CoA reductase and L-arginine lowers LDL cholesterol by about 2 to about 20 mg/dL more compared to administration of HMG-CoA reductase inhibitor alone. In yet another embodiment, the coadministration of HMG-CoA reductase and L-arginine lowers triglycerides by about 5 to about 50 mg/dL, or alternatively by about 20 to about 35 mg/dL, more compared to administration of HMG-CoA reductase inhibitor alone.

In another embodiment, the methods of the present invention may be used to lower C-reactive protein in a subject. C-reactive protein is an acute phase reactant released by the body in response to acute injury, infection, or other inflammatory stimuli. Studies have demonstrated a positive correlation between C-reactive protein and coronary artery disease. Ridker, Circulation 108(12): e81-85 (2003); Blake et al., Am. J. Physiol. Regul. Integr. Comp. Physiol. 285(5): R1250-1252 (2003). In one embodiment, the methods lower C-reactive protein by about 10% to about 50%, or by about 25% to about 35%.

The coadministration of HMG-CoA reductase inhibitor and L-arginine has a synergistic effect in lowering C-reactive protein. In one embodiment, the method lowers C-reactive protein by about 50% to about 90%, or about 65% to about 75%, more compared to administration of HMG-CoA reductase inhibitor without the sustained release formulation of L-arginine. In another embodiment, the method lowers C-reactive protein by about 80% to about 120%, or about 95% to about 105%, more compared to administration of the sustained release formulation of L-arginine without HMG-CoA reductase inhibitor.

Furthermore, methods of the present invention may be used to increase nitric oxide production and/or increase vasodilation in a subject with elevated asymmetrical dimethylarginine (ADMA). Asymmetrical dimethylarginine (ADMA) is an endogenous, competitive inhibitor of eNOS. The presence of elevated plasma ADMA levels is associated with endothelial dysfunction. Statins stimulate the expression of endothelial NO synthase (eNOS) in vitro and enhance endothelium-dependent, NO-mediated vasodilation in vivo. Accordingly, statins (e.g., simvastatin) can enhance endothelial function in patients with elevated ADMA. Without wishing to be bound by theory, it is believed that the inhibitory effect of ADMA is overcome by L-arginine.

By administering L-arginine, and, optionally, an HMG-CoA reductase inhibitor (e.g., simvastatin), to a subject with elevated ADMA, the methods of the present invention can increase nitric oxide production and/or increase vasodilation. Such coadministration can increase endothelial function by about 5% to about 15% or alternatively, by about 7% to about 12%. In one embodiment according to the invention, the subject has endothelial dysfunction.

For any mode of administration, the actual amount of compound delivered, as well as the dosing schedule necessary to achieve the advantageous pharmacokinetic profiles described herein, will depend, in part, on such factors as the bioavailability of the compound (and/or an active metabolite thereof), the disorder being treated, the desired therapeutic dose, and other factors that will be apparent to those of skill in the art. The actual amount delivered and dosing schedule can be readily determined by those of skill without undue experimentation by monitoring the blood plasma levels of administered compound and/or an active metabolite thereof, and adjusting the dosage or dosing schedule as necessary to achieve the desired pharmacokinetic profile.

The formulations used in the methods of the invention, as described herein, or pharmaceutically acceptable addition salts or hydrates thereof, can be delivered to a subject so as to avoid or reduce undesirable side effects according to the invention using a wide variety of routes or modes of administration. In one embodiment, the subject is an animal. In another embodiment, the subject is a mammal. In yet another embodiment, the subject is a human. The most suitable route in any given case will depend on the nature and severity of the condition being treated. The preferred route of administration of the present invention is the oral route. The compositions may be conveniently presented in unit dosage form, and prepared by any of the methods well known in the art of pharmacy. Techniques and formulations for administering the compositions may be found in Remington: the Science & Practice of Pharmacy, by Alfonso R. Gennaro, 20th ed., Williams & Wilkins, 2000.

The formulations of the invention will generally be used in an amount effective to achieve the intended purpose, e.g., to treat and/or prevent a cerebrovascular and/or cardiovascular disease or disorder. By therapeutically effective amount is meant an amount effective to treat a disease, disorder, symptom related to a disease or disorder, or predisposition toward a disease or disorder. As described earlier, the term “treat” refers to the application or administration of a therapeutic agent or formulation to a patient, or application or administration of a therapeutic agent or formulation to an isolated tissue from a patient, who has a disease or disorder, a symptom of disease or disorder or a predisposition toward a disease or disorder, with the purpose of curing, healing, alleviating, relieving, altering, remedying, ameliorating, delaying onset of the disease or disorder and/or event, slowing the progression of the disease or disorder, improving or affecting the disease or disorder, the symptoms of disease or disorder or the predisposition toward a disease or disorder and/or event. Determination of a therapeutically effective amount is well within the capabilities of those skilled in that art, especially in light of the detailed disclosure provided herein.

Pharmaceutical formulations suitable for use with the present invention include formulations wherein L-arginine and/or an HMG-CoA reductase inhibitor are contained in a therapeutically effective amount, i.e., an amount effective to achieve the intended purpose. In general, an effective amount is that amount of a pharmaceutical preparation that alone, or together with further doses, produces the desired response. This may involve only slowing the progression of the disease temporarily. In another embodiment, it involves halting the progression of the disease permanently or delaying the onset of or preventing the disease or condition from occurring. The effect of the dosage on any particular disease can be monitored by routine methods. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner.

Generally, doses of active compounds would be from about 0.01 mg/kg per day to about 1000 mg/kg per day. In one embodiment, it is expected that doses ranging from about 50 to about 500 mg/kg will be suitable. In another embodiment, administration is oral and in one or several administrations per day.

In another embodiment, the subject will receive less than about 10 g sustained release L-arginine per day for example, less than about 9 g, about 8 g, about 7 g, about 6 g, about 5 g, about 4 g, about 3 g, about 2 g, or about 1 g sustained release L-arginine per day. For example, the subject may receive a daily dosage of from about 1 g to about 7 g, about 2 g to about 6 g or about 3 g to about 5 g sustained release L-arginine. Ranges of values using a combination of any of the above recited values as upper and/or lower limits are intended to be included. Preferably, the subject receives less than about 4 g sustained release L-arginine per day. While not risking to be bound by theory, the sustained release formulations of L-arginine allow for a small dosage to be employed, i.e. the total amount of L-arginine may be lower and yet still achieve a therapeutic effect.

Of course, the actual amount of L-arginine and/or an HMG-CoA reductase inhibitor will depend on, among other things, the condition of the subject, and the weight and metabolism of the subject. For example, when administered to a subject suffering from IC or AD, a tablet, pill, dragee, capsule, gelcap, troche, or capsule, will contain an amount of L-arginine and/or an HMG-CoA reductase inhibitor effective to, inter alia, ameliorate the harmful effects of insufficient blood flow to normal tissue, i.e., prevent the development of or alleviate the existing symptoms of, or prolong the survival of, the subject being treated. Determination of an effective amount is well within the capabilities of those skilled in the art, especially in light of the detailed disclosure herein.

Therapeutically effective amounts for use in humans can also be estimated from animal models. For example, a dose for humans can be formulated to achieve a concentration found to be effective in animals.

A therapeutically effective dose can also be estimated from human pharmacokinetic data. While not intending to be bound by any particular theory, it is believed that efficacy is related to a subject's total exposure to an applied dose of administered drug, and/or an active metabolite thereof, as determined by measuring the area under the blood concentration-time curve (AUC). Thus, a dose administered according to the methods of the invention that has an AUC of administered compound (and/or an active metabolite thereof) within about 50% of the AUC of a dose known to be effective for the indication being treated is expected to be effective. A dose that has an AUC of administered compound (and/or an active metabolite thereof) within about 70%, about 80% or even about 90% or more of the AUC of a known effective dose is preferred. Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Formulations that exhibit large therapeutic indices are preferred. While formulations that exhibit toxic side effects may be used, care should be taken to design a delivery system that targets such formulations to the site of affected tissue in order to minimize potential damage to uninfected cells and, thereby, reduce side effects.

The data obtained from the cell culture assays and animal studies can be used in formulating a range of dosage for use in humans. In one embodiment, the dosage of such formulations of the instant invention lies within a range of circulating concentrations that include the ED50 with little or no toxicity. The dosage may vary within this range depending upon the dosage form employed and the route of administration utilized. For any formulation used in the therapeutic or prophylactic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Adjusting the dose to achieve maximal efficacy in subjects based on the methods described above, particularly on the blood concentration and duration of administered compound and/or its active metabolites is well within the capabilities of the ordinarily skilled artisan.

III. Methods of Manufacture

It has been discovered that efficient and substantial incorporation or coverage of L-arginine granules within a matrix improves the sustained release characteristics of the compositions of the present invention. In the case of a cellulosic matrix, upon contact with water, the matrix is partially hydrated, forming a gel layer that controls the rate of release of the L-arginine. Efficient coating or incorporation of the L-arginine granules creates a temporary barrier to dissolution that prolongs the delivery of the L-arginine. Substantial gaps in the matrix allow the L-arginine to dissolve too quickly. The methods of the present invention result in a product with improved properties versus products made by direct compaction. Further, the present method is advantageous over methods that include fluidization dispersions as these methods are time-consuming and expensive.

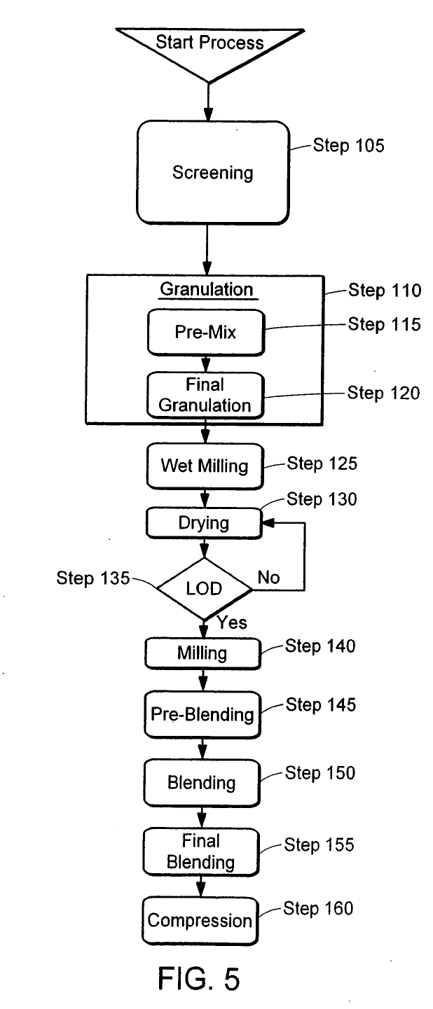

The key to effective and efficient coverage is in performing the granulating, milling, and blending steps of the present invention. Referring to FIG. 5, in a preferred embodiment, tablets are manufactured according a method that includes the steps of granulating the L-arginine (step 110), milling the L-arginine (steps 125, 140), blending the L-arginine with the remainder of the ingredients (steps 145, 150, 155), and compressing the ingredients to form a tablet (step 160). Preferably, the method also includes either or both of the steps of screening the ingredients (step 105), and/or drying the L-arginine during the milling step (step 135).

If the ingredients are screened prior to use (step 105), a #20 and/or a #30 mesh screen can be used for some or all of the ingredients. In a preferred embodiment, the granules are screened before granulation (step 105), and again before milling (not shown). Screening provides granules with a narrower particle size distribution in a range that is advantageous for coating and/or compaction.

The step of granulating is advantageous in that it provides more uniform particles. An active agent can be pelletized or granulated using any suitable methods known in the art. Pelletization or granulation is commonly defined as a size-enlargement process in which small particles are gathered into larger, permanent aggregates in which the original particles can still be identified and renders them into a free flowing state. Prior to granulation, a binder can be added to the active agent to improve the granulation process. Other additives can be added during granulation. These include, e.g., sweeteners, flavors, color agents, antioxidants, etc.

Optionally, water or other solvent can be added to aid the granulation process. The amount of water or solvent added depends on, for example, the selection of a granulation process, and is readily determinable by those of skill in the art. Water or other solvent may be added at any suitable time point during the granulation process. For example, a binder may be mixed with a solvent (e.g., water) to form a granulating agent, and then the granulating agent can be sprayed onto active agents. Alternatively, if a granulating agent is too viscous to be uniformly sprayed onto active agents, it may be desirable to blend the binder with the active agent first and then spray water or other solvent to produce a uniform pattern of active agent granules or pellets.

Any suitable granulation method can be used to produce particles comprising an active agent. Wet granulation and/or dry granulation methods can be used.

Dry granulation refers to the granulation of a formulation without the use of heat and solvent. Dry granulation technology generally includes slugging or roll compaction. Slugging consists of dry-blending a formulation and compressing the formulation into a large tablet or slugs on a compressing machine. The resulting tablets or slugs are milled to yield the granules. Roller compaction is similar to slugging, but in roller compaction, a roller compactor is used instead of the tableting machines. See, e.g. Handbook of Pharmaceutical Granulation Technology, D. M. Parikh, eds., Marcel-Dekker, Inc. pages 102-103 (1997). The dry granulation technique is useful in certain instances, for example, when the active agent is sensitive to heat or solvent.

Alternatively, wet granulation can be used. In wet granulation, solvents and binders are typically added to a formulation to provide larger aggregates of granules. The temperature during granulation can be set at any suitable point, generally not exceeding the melting point of any components of the formulation. Typically, the mixture is granulated at a temperature of about 35° C. to about 65° C. for about 20 to about 90 minutes. In a preferred embodiment, the mixture is granulated for less than about 20 minutes, more preferably for about 1 to about 10 minutes at room temperature (see, Example 8). Then the granules are typically air dried for a suitable duration (e.g., one or more hours).

Preferably, the active agents are granulated by high shear mixer granulation (“HSG”) or fluid-bed granulation (“FBG”). Both of these granulation processes provide enlarged granules or pellets but differ in the apparatuses used and the mechanism of the process operation. These granulation techniques can be performed using commercially available apparatuses.

In HSG, blending and wet massing are accomplished by high mechanical agitation by an impeller and a chopper. Mixing, densification, and agglomeration of wetted materials are achieved through shearing and compaction forces exerted by the impeller. The primary function of the chopper is to cut lumps into smaller fragments and aid the distribution of the liquid binder. The liquid binder is either poured into the bowl or sprayed onto the powder to achieve a more homogeneous liquid distribution.

On the other hand, fluidization is the operation by which fine solids are transformed into a fluid-like state through contact with a gas. At certain gas velocities, the fluid will support the particles, giving them freedom of mobility without entrainment. Such a fluidized bed resembles a vigorously boiling fluid, with solid particles undergoing extremely turbulent motion, which increases with gas velocity. Fluidized bed granulation is thus a process by which granules are produced in a fluidized bed by spraying a binder solution onto a fluidized powder bed to form larger granules. The binder solution can be sprayed from, for example, a spray gun positioned in any suitable manner (e.g., top or bottom). The spray position and the rate of spray may depend on the nature of the active agent and the binder used, and are readily determined by those skilled in the art.

In a preferred method according to the invention, granulating the L-arginine (step 110) includes the steps of premixing the L-arginine with a binder such as povidone to form a blend (step 115), and granulating the blend with a granulating agent (granulating vehicle) in a granulator (step 120). The granulating agent can be, e.g., povidone dissolved in purified water. Preferably, a high-shear granulator such as a Niro PMA 65 High Shear Granulator is employed. The granulator can be used both to mix the L-arginine and binder, and also to granulate the blend while spraying the granulating vehicle on the blend.

After the granulation of one or more components of the formulation, optionally, the granulated formulation can be milled. Milling can be performed using any suitable commercially available apparatus (e.g., CoMil equipped with a 0.039 inch screen). The mesh size for the screen can be selected depending on the size of the granules desired. After the granulated active agents are milled, they may be further dried (e.g., in the air) if desired.

In a preferred embodiment, milling the L-arginine includes the steps of milling the wet granules or wet milling (step 125), drying the granules (step 130), and milling the dry granules or dry milling (step 140), in accordance with techniques well known in the art (see generally, U.S. Pat. No. 5,145,684 and European Patent Application 498,482, the contents of both of which are hereby incorporated by reference). A mill such as a CoMil can be employed to wet mill and dry mill the granules. In one embodiment, the mill is equipped with a '375Q screen for wet milling and a '062R screen for dry milling. The drying step can be accomplished by drying the granules in a bed dryer, e.g., an Aeromatic S-2 Fluid Bed Dryer, to a desired Loss on Drying (LOD) level, e.g., a ≦3% LOD. The drying steps can be accomplished in stages (step 135) until the desired LOD is reached.

Blending the L-arginine with the remainder of the ingredients can include a pre-blending step (step 145), a blending step (step 150), and a final blending step (step 155). The pre-blending step can include blending the L-arginine/povidone granules with a filler and a glidant, e.g., microcrystalline cellulose and colloidal silicon dioxide. The pre-blending step can be accomplished, e.g., in an 8 quart V-Blender, by blending for about 5 minutes at 25 rpm. The blending step can include adding to this blend one or more sustained release agents, e.g., one or more hydroxypropyl methylcelluloses, and a filler, e.g., microcrystalline cellulose. The blending step can be accomplished, e.g., in a 2 cubic foot V-Blender, by blending for about 20 minutes at 25 rpm. The final blending step can include adding a release agent/lubricant, e.g., magnesium stearate, to the blend in the 2 cubic foot V-blender and blending for about 5 minutes at 25 rpm.

After preparing the formulation as described above, the formulation is compressed (step 160) into a tablet form. This tablet shaping can be done by any suitable means, with or without compressive force. For example, compression of the formulation after the granulation step can be accomplished using any tablet press (e.g., a Manesty Beta Press equipped with a 0.748″×0.380″ oval shaped, convex, plain tooling), preferably if the formulation composition is adequately lubricated with lubricant (e.g., magnesium stearate). Many alternative means to effect this step are available, and the invention is not limited by the use of any particular apparatus. The compression step can be carried out using a rotary type tablet press. The rotary type tableting machine has a rotary board with multiple through-holes, or dies, for forming tablets. The formulation is inserted into the die and is subsequently press-molded.

Alternatively, the tablets can be made by molding. Molded tablets may be made by molding in a suitable machine a mixture of the powdered compound moistened with an inert liquid diluent.

The diameter and shape of the tablet depends on the molds, dies and punches selected for the shaping or compression of the granulation composition. Tablets can be discoid, oval, oblong, round, cylindrical, triangular, and the like. The tablets may be scored to facilitate breaking. The top or lower surface can be embossed or debossed with a symbol or letters.

The compression force can be selected based on the type/model of press, what physical properties are desired for the tablet product (e.g., desired hardness, friability, etc.), the desired tablet appearance and size, and the like. Typically, the compression force applied is such that the compressed tablets have a hardness of at least about 2 kp. These tablets generally provide sufficient hardness and strength to be packaged, shipped or handled by the user. If desired, a higher compression force can be applied to the tablet to increase the tablet hardness. However, the compression force is preferably selected so that it does not deform (e.g., crack or break) the active agent-containing particles within the tablet. Preferably, the compression force applied is such that the compressed tablet has a hardness of less than about 10 kp. In certain embodiments, it may be preferred to compress a tablet to a hardness of between about 3 kp to about 7 kp, optionally between about 3 kp to about 5 kp, or about 3 kp.

Typically, the final tablet will have a weight of about 50 mg to about 2000 mg, more typically about 200 mg to about 1000 mg, or about 400 mg to about 700 mg.

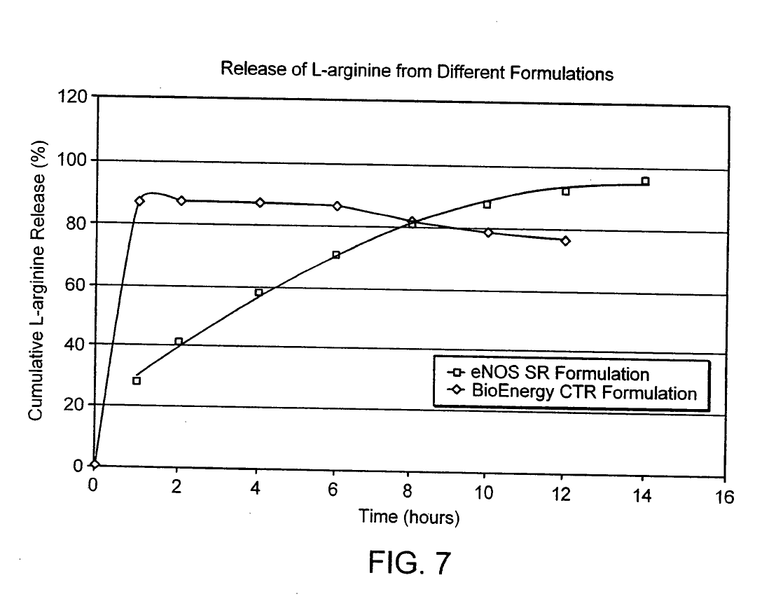

The particular formulation and methods of manufacturing the formulation of the present invention impart unique advantages on the sustained release L-arginine composition. In particular, the formulation and the methods of the present invention render a composition that achieves a desirable sustained release dissolution profile. Optimally, a sustained release L-arginine formulation would sustain in vitro drug release at least up to 14 hours, preferably about 10% to about 40% at about 1 hour, about 30% to about 70% at about 4 hours, about 55% to about 75% at about 6 hours, about 65% to about 85% at about 8 hours, about 75% to about 95% at about 12 hours and about 80% to about 100% at 14 hours. As demonstrated by FIG. 7, the formulation of the present invention achieves such optimal dissolution. Furthermore, as shown in Example 8 and Example 14, dissolution and stability studies demonstrate that the formulation of the present invention displays an optimal dissolution profile one and two months following manufacturing.

Furthermore, the formulation and methods of the present invention render a sustained release L-arginine composition that is not excessively friable. Furthermore the formulation and methods of the present invention render a sustained release L-arginine composition that is sufficiently compressible to allow for convenient manufacturing of the composition.

If desired, other modifications can be incorporated into embodiments of the tablet. For example, modification of active agent release through the tablet matrix of the present invention can also be achieved by any known technique, such as, e.g., application of various coatings, e.g., ion exchange complexes with, e.g., Amberlite IRP-69. The tablets of the invention can also include or be coadministered with GI motility-reducing drugs. The active agent can also be modified to generate a prodrug by chemical modification of a biologically active compound that will liberate the active compound in vivo by enzymatic or hydrolytic cleavage, etc. Additional layers or coating can act as diffusional barriers to provide additional means to control rate and timing of drug release.

If an HMG CoA-reductase inhibitor (e.g., simvastatin) and/or additional agents are included, preferably these agents are added in the blending steps (steps 145, 150, 155). When the tablet comprises a sustained release L-arginine formulation and an HMG-CoA reductase inhibitor formulation, the tablet may have a core of slow release L-arginine formulation and a second outer cover or coating of a formulation comprising at least one HMG-CoA reductase inhibitor. Alternatively, the tablet may comprise an L-arginine formulation, e.g., a sustained release L-arginine formulation, and a HMG-CoA reductase inhibitor formulation sharing one surface.

When L-arginine is administered either sequentially or concurrently with HMG-CoA reductase inhibitors, each tablet, cachet, troche, or capsule contains from about 0.01 mg to about 200 mg of the HMG-CoA reductase inhibitors. The amount of an HMG-CoA reductase inhibitor will vary depending on the particular HMG-CoA reductase inhibitor utilized.

In another aspect of the present invention, a composition for the treatment of cardiovascular and/or cerebrovascular disease is provided in the form of food. Preferably, the food is in the form of a bar such as a prescription health bar. Use of food enables the provision of larger amounts of L-arginine than could be incorporated into a single tablet, e.g., it is difficult to incorporate more than 1 gram of L-arginine in a single tablet. Thus, multiple tablets are required for delivery of amounts of L-arginine in excess of 1 gram. The present invention provides a bar that can provide more than 1 gram of L-arginine as well as other agents, as desired. In one embodiment, the L-arginine is added as an immediate release formulation, e.g., immediate release granulars of L-arginine, to a food bar. Preferably, the bar includes a sustained release formulation that includes, for example, sustained release granulars of L-arginine. In a preferred embodiment, the granulars include taste masking constituents, e.g., taste making coatings. In another embodiment, the bar further contains additional agents, such as an HMG-CoA reductase inhibitor. Preferably, the HMG-CoA reductase inhibitor, is a statin such as simvastatin. Combining L-arginine with statins in a food vehicle form would provide continence and an easy to administer the formulation. Use of food also can reduce the need for taking multiple tablets of L-arginine when a higher dose is desired.

In one embodiment, the bars have between about 1 and about 80 g of simvastatin and between about 1 and about 10 grams of L-arginine. In a preferred embodiment, bars are provided having a total of at least about 10 mg of simvastatin and about 4 g per bar of L-arginine or its salts in conjunction with sugars, fruit components, protein, and vitamins and minerals. The bar weighs in the range of about 25 to about 100 g. In a particular process, the bar is produced by combining sugars and fruit paste at an elevated temperature and then combining the syrup at a reduced temperature with the minor ingredients. After blending the minor ingredients in the syrup, the L-arginine and the simvastatin are added, particularly in conjunction with a protein extender, followed by bulking and food agents, particularly fruit pieces or other particulate edible ingredients providing the desired texture and flavor, and soy proteins. The resulting product is storage stable, has desirable organoleptic properties in being tasty, and provides a healthy combination of ingredients in collaboration with the simvastatin and L-arginine. Methods and formulations for manufacturing health bars with L-arginine and L-lysine are described in, e.g., U.S. Pat. No. 6,063,432, incorporated in its entirety by this reference.

Another aspect of the present invention is a method of manufacturing the bar described above. The method would include granulating the L-arginine as described above in connection with FIG. 5, step 110. Preferably the granulating step would include the pre-mixing step (step 115) and the granulating step (step 120). Preferably, the method also includes the wet milling step (step 125) described above. Such bar would be obtained by wet granulation of the L-arginine with appropriate excipients, such as detailed above. The resulting granulars would be either used as is or be coated with taste masking cellulosics.

This invention is further illustrated by the following examples that should not be construed as limiting. The contents of all references, patents and published patent applications cited throughout this application are incorporated herein by reference.

### EXAMPLES

### Example 1

### Tablet Formulation 1

About 250 grams of L-arginine was placed in a mixer and as it was slowly mixed at 100 RPM, 100 g EUDRAGIT RS 30D low permeability methacrylic aqueous polymer dispersion (Rohm America, Piscataway, N.J.) was added to form a wet mass. The wet mass was passed through 18-20 sieves and allowed to dry at 50° C. for 24 hours. The resulting dry L-arginine granulars (250 g) were dry mixed with 84 g METHOCEL K100 M CR methylcellulose (The Dow Chemical Company, Danbury, Conn.) and 3 g magnesium stearate to form a blend. The resulting blend was compressed into tablets using 7/16 concave punches.

### Example 2

### Tablet Formulation 2

250 g of L-arginine was placed in a mixer and as it was slowly mixed, 84 g METHOCEL K100 M CR methylcellulose and 3 g magnesium stearate were added. The resulting blend was compressed into tablets using 7/16 concave punches.

### Example 3

### Capsule Formulation 1

250 g L-arginine was placed in a mixer and as it was slowly mixed, 100 g EUDRAGIT RS 30D low permeability methacrylic aqueous polymer dispersion was added to form a wet mass. The wet mass was passed through 18-20 sieves and allowed to dry at 50° C. for 24 hours. The resulting dry L-arginine granulars (250 g) were dry mixed with 84 g METHOCEL K100 M CR methylcellulose and 3 g magnesium stearate to form a blend. The resulting blend was placed into 00 gel capsules.

### Example 4

### Capsule Formulation 2

250 g L-arginine was placed in a mixer and as it was slowly mixed, 84 g METHOCEL K100 M CR methylcellulose and 3 g magnesium stearate were added. The resulting blend was placed into 00 gel capsules.

### Example 5

### Tablet Formulation 3

250 g L-arginine and 50 g METHOCEL K100 M CR methylcellulose were mixed and homogenized using a Kitchen Aid® mixer on low speed for 10 minutes to form a dry blend. To the dry blend, 115 g EUDRAGIT RS 30D low permeability methacrylic aqueous polymer dispersion was added in 5 g increments until the mass was homogeneously wet. The wet mass was passed through a 12 mesh sieve followed by a 20 mesh sieve and subsequently, allowed to dry at 30° C. for 24 hours until the moisture content was 1% by weight. The resulting dry L-arginine granulars were dry-mixed with 7 g magnesium stearate and then compressed, using a Beta Manesy press, into tablets using 7/16 concave punches.

### Example 6

### Manufacturing of a Sustained Release Tablet

About 1000 g L-arginine and about 200 g METHOCEL K100 M CR methylcellulose were mixed in a GP-1 high shear mixer (granulator) for about 5 minutes at 100 RPM. About 138 g EUDRAGIT RS 30D low permeability methacrylic aqueous polymer dispersion was then added with the impeller running at 200 RPM and a pressure of 1.5 bar. The mixture was granulated for 1 minute at 200 RPM. The granulation was then dried in an MP-1 Fluid Bed Granulator at 45° C. inlet temperature with an air flow of 100 CMH to approximately 2% moisture content. The dried granules were then milled using a Comil 197S with size 55R screen and round impeller at 90% speed. In an 8 Qt. V-Blender, about 27 g magnesium stearate was added to the milled granules and mixed for 2 minutes. The material was then compressed into tablets with a target weight of 682.5 mg to highest possible hardness using a Beta Manesty Press with 7/16″ standard concave tooling. The tablets were hand-packaged at 60 tablets per bottle in 75 cc HDPE Bottles.

The release profile of the tablet versus commercially available sustained release L-arginine tablets purchased from BioEnergy (Warren, N.J.), was generated using high performance liquid chromatography (HPLC). FIG. 7 is a chart depicting the release profiles of both formulations.

### Example 7

### Evaluation of Pharmacokinetics of L-arginine

A randomized, four-way crossover study to evaluate the pharmacokinetics of L-arginine sustained release tablets versus immediate release capsules was conducted on 14 healthy adult volunteers under fasting conditions. “Healthy” as used herein means nonhypercholestermic subjects with no cardiovascular risk factors. The study compared the sustained release L-arginine tablet (L-arginine SR) of Example 6 and commercially available immediate release L-arginine capsules (L-arginine IR) purchased from Montiff (Los Angeles, Calif.).

The study goal was to determine the pharmacokinetic parameters of sustained release L-arginine. As depicted in Table I below, based on the p-values from the two-tailed paired t-test performed on each pharmacokinetic parameters, there was a statistically significant difference between treatments for Cₘₐₓ and Tₘₐₓ. As expected, sustained release L-arginine tablets had a lower Cₘₐₓ (14.9 ug/mL versus 24.1 ug/mL) and a longer Tₘₐₓ (4.4 h versus 1.4 h) compared with the immediate release capsules.

TABLE I
PK Parameters of L-arginine SR v. L-arginine IR

| L-arginine | Cₘₐₓ | AUC₀₋ₜ | AUC₀₋₁₀ | T_{max 0-t} | T_{max 0-10} |
|---|---|---|---|---|---|
| L-Arg SR | 14.9 | 143 | 68.56 | 4.4 | 3.27 |
| L-Arg IR | 24.1 | 147 | 92.23 | 1.4 | 1.35 |
| % Ratio | 0.62 | 0.97 | 0.74 | 3.2 | 2.43 |
| P-value | 0.0005 | 0.677 | 0.0382 | 0.0133 | 0.0073 |

### Example 8

### Manufacturing of an Improved Sustained Release L-arginine Tablet

Table II lists the ingredients assembled to manufacture an improved sustained release tablet, as well as the amounts used of each ingredient.

TABLE II
Ingredients

| | | Percentage | Weight/Batch |
|---|---|---|---|
| Component | mg/tablet | (%) | (Kg) |
| L-arginine monohydrochloride | 500 | 50 | 12.5 |
| Povidone (K 29/32) | 35 | 3.5 | 0.88 |
| Purified Water | — | — | 2* |
| Hydroxypropyl Methylcellulose | 275 | 27.5 | 6.87 |
| (METHOCEL K100M P CR) | | | |
| Hydroxypropyl Methylcellulose | 75 | 7.5 | 1.88 |
| (METHOCEL E 4M CR) | | | |
| Microcrystaline Cellulose | 102.5 | 10.2 | 2.56 |
| (AVICEL PH 102) | | | |
| Colloidal Silicon Dioxide | 5 | 0.5 | 0.13 |
| Magnesium stearate | 7.5 | 0.75 | 0.18 |
| TOTAL: | 1000 | 100.0 | 25 |

*Water is used in granulation and then the mixture was dried

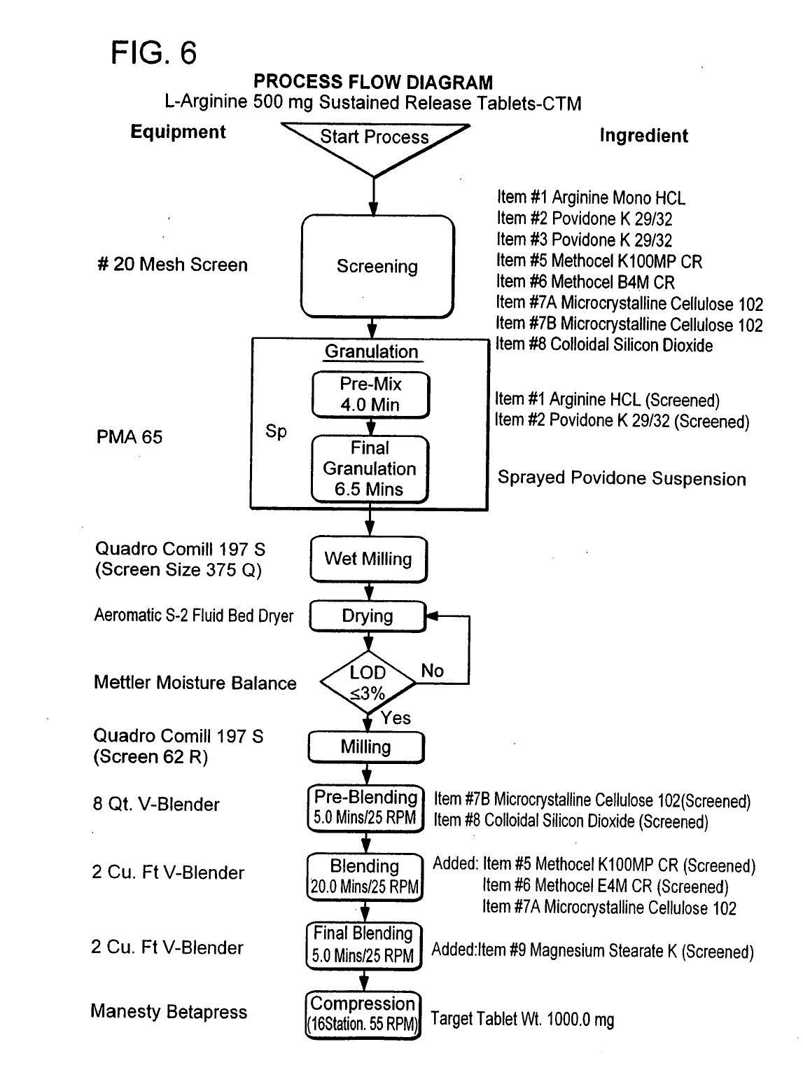

All ingredients, except the magnesium stearate, were screened in a #20 mesh screen. The magnesium stearate was screened in a #30 mesh screen. Approximately half of the povidone (polyvinylpyrrolidone) was dissolved in purified water and set aside as a granulating agent. The L-arginine and the remainder of the povidone were dry mixed for 4 minutes in a Niro PMA 65 High Shear Granulator, and then granulated for about 6.5 minutes by spraying the granulating agent into it. The wet granules were then milled in a CoMil mill equipped with a '375Q screen. The milled granules were then dried in an Aeromatic S-2 Fluid Bed Dryer to a LOD of ≦3%. The dried granules were then milled in the CoMil equipped with a '062R screen. Approximately half of the microcrystalline cellulose and the collodial silicon dioxide were then blended in an 8 quart V-Blender for 5 minutes at 25 rpm and transferred to a 2 cubic foot V-Blender. The remaining portion of the microcrystalline cellulose and the hydroxylpropyl methylcellulose were then also added to the 2 cubic foot V-Blender and blended for 20 minutes at 25 rpm. The magnesium stearate was then added to the 2 cubic foot V-Blender and blended for 5 minutes at 25 rpm. Finally, the blend was compressed into tablets with a target weight of 1000 mg using a Manesty Bet Press equipped with 0.748″×0.380″ oval shaped, convex, plain tooling. FIG. 6 is a schematic flow diagram of this method.

Standard in-process controls tests and specifications can be used during the manufacturing process, the ones used for this example are listed in Table III below.

TABLE III
L-arginine SR Tablets In-process Controls:
Specifications and Methods

| Specification: | Method | Acceptance Criteria |
|---|---|---|
| Blend Uniformity | CTMLP-663 | Mean: 90.0%-110.0% |
| | | of Label Claim |
| | | RSD % NMT 5.0% |
| Bulk & Tap Density | SOP Lab 2010 | Report results |
| Particle Size | SOP LAB 2018 | Report results |
| Distribution | | |
| Moisture | SOP Lab 2059 | NMT 3.5% |

Standard release methods and specifications can be used, the ones used for this example are provided in Table IV below.

TABLE IV
L-arginine SR Tablets Release Methods and Specifications

| Specification: | Method | Acceptance Criteria |
|---|---|---|
| Physical | Visual | White to off-white tablets |
| Appearance | Inspection | Oval shaped, convex tablet |
| Identification | CTMPLP-663 | The retention time and on-line |
| | | UV spectrum (200-400 nm) of |
| | | the sample, correspond to those |
| | | of the reference standard |
| Potency | CTMLP-663 | 90.0-110.0% of label claims |
| Related Substances | CTMLP-663 | Individual: NMT 0.5% |
| | | Total: NMT 2.0% |
| Moisture | SOP LAB 2059 | NMT 3.5% |
| Dissolution Profile | CTMLP-663 | 1 hr 10-40% |
| | | 4 hr 30-70% |
| | | 12 hr ≧75% |
| | | Record Profile |
| Content Uniformity | CTMLP-663 | USP <905> |
| Microbial Limits | USP <61> | Total Aerobic Microbial |
| | | count ≦100 cfu/mL |
| | | Total Combined Molds and Yeast |
| | | count ≦50 cfu/mL |
| | | Absence of E. coli |
| | | Absence of S. aureus |
| | | Absence of P. aeruginosa |
| | | Absence of Salmonella species |

Furthermore, the studies have demonstrated desirable physical characteristics, including friability and content uniformity for the sustained release L-arginine formulations of the present invention.

TABLE V
Physical Testing, Potency, Content Uniformity and Dissolution
for Two batches of the SR L-arginine formulation

Batch #

| | 1 | 2 |
|---|---|---|
| Average tablet weight n = 20, mg | 1003.3 | 1014.5 |
| Tablet hardness n = 20, kp | 11.0 | 12.4 |
| Tablet thickness n = 20, mm | 7.89 | 7.70 |
| Tablet friability, % | 0.1 | 0.1 |
| Potency, % | 98.4 | 100.5 |
| Content Uniformity n = 10; % | 99.0 | 100.8 |
| Content Uniformity, % RSD | 1.5 | 1.8 |

| | |
|---|---|
| Dissolution Time, hr | % Release |

| | | |
|---|---|---|
| 0 | 0 | 0 |
| 1 | 27.3 | 26.8 |
| 2 | 42.1 | 42.1 |
| 4 | 59.9 | 60.2 |
| 6 | 73.0 | 73.6 |
| 8 | 82.8 | 83.4 |
| 10 | 90.3 | 90.3 |
| 12 | 95.1 | 94.9 |
| 14 | 98.4 | 92.5 |

### Example 9

### Evaluation of Pharmacokinetics of L-arginine SR with and without Simvastatin and Simvastatin with and without L-arginine SR

The pharmokinetics of L-arginine SR with and without simvastatin, and simvastatin with and without L-arginine SR were studied. The L-arginine SR tablets of Example 6 were used as well as commercially available simvastatin tablets purchased from BioEnergy (Warren, N.J.).

As can be see in Table VI, based on the p-values from the two-tailed paired t-test performed on each pharmokinetic parameter, there was not a statistically significant difference between treatments for Cₘₐₓ, AUCO₀₋₁₀, and Tₘₐₓ. As depicted in Table VII, L-arginine SR has no statistically significant effect on the single dose pharmokinetics of simvastatin.

TABLE VI
L-arginine PK Paramaters with and without Simvastatin

| | Cₘₐₓ | AUC₀₋₁₀ | Tₘₐₓ |
|---|---|---|---|
| L-arginine | (mg/ml) | (mg-hr/ml) | (hr) |
| L-Arg SR | 14.77 | 68.56 | 3.27 |
| L-Arg SR with | 13.49 | 51.55 | 3.23 |
| Simvastatin | | | |
| % Ratio | 1.09 | 1.33 | 1.01 |
| P-value | 0.5001 | 0.0713 | 0.9716 |

TABLE VII
Simvastatin PK Paramaters with and without L-arginine

| | Cₘₐₓ | AUC₀₋₁₀ | Tₘₐₓ | kₑₗᵢₘ | t_{1/2} |
|---|---|---|---|---|---|
| Simvastatin | (ng/ml) | (ng-hr/ml) | (hr) | (1/hr) | (hr) |
| simvastatin w/o | 21.15 | 107.93 | 2.68 | 0.1248 | 6.56 |
| L-arginine SR | | | | | |
| simvastatin with | 18.95 | 114.36 | 2.29 | 0.0950 | 10.01 |
| L-arginine SR | | | | | |
| P-value | 0.5360 | 0.6302 | 0.4758 | 0.1526 | 0.1059 |

### Example 10

### Effect of Administration of Simvastatin with L-arginine Upon Infarct Size in Mice

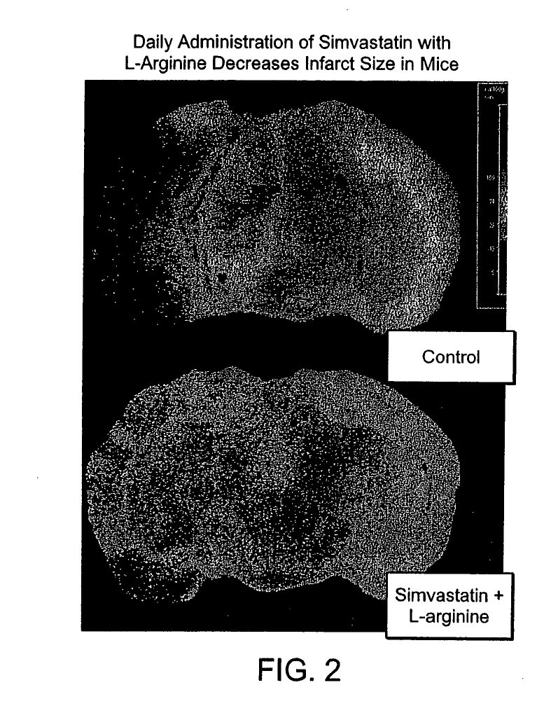

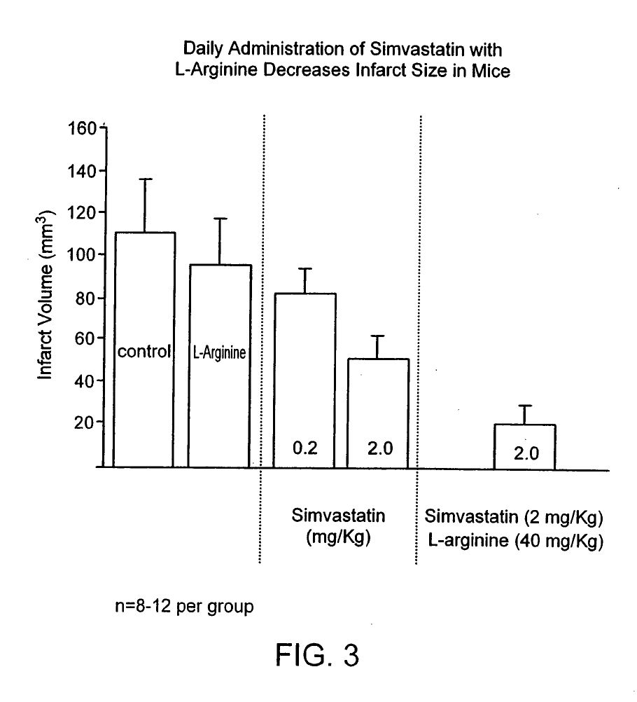

The effect of administration of both simvastatin and L-arginine upon infarct size was studied in mice. Mice were given interperitoneal injections comprising simvastatin, and simvastatin and L-arginine, dissolved in saline solution in the amounts indicated in FIG. 3. The results of infarct size on these mice versus a control group are depicted in FIG. 2 and FIG. 3.

### Example 11

### Dose Optimization of Combination of Simvastatin and L-arginine

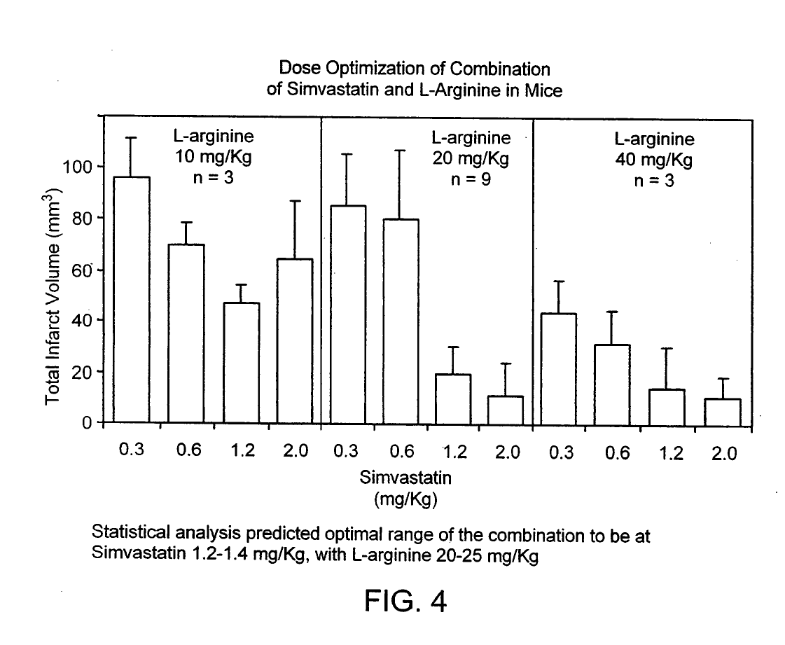

Dose optimization of combined administration of simvastatin and L-arginine was studied in mice. Mice were injected with varying levels of simvastatin and L-arginine as shown in FIG. 4. The results of this study are also shown in FIG. 4. Statistical analysis predicted that the optimal range of the combination to be 1.2-1.4 mg/Kg simvastatin with about 20-25 mg/Kg L-arginine.

### Example 12

### Improvement of Endothelium-Dependent Vasodilation by Simvastatin is Potentiated by Combination with L-arginine Sustained Release in Patients with Elevated ADMA Levels

Statins stimulate the expression of endothelial NO synthase (eNOS) in vitro and enhance endothelium-dependent, NO-mediated vasodilation in vivo. Asymmetrical dimethylarginine (ADMA) is an endogenous, competitive inhibitor of eNOS. The presence of elevated plasma ADMA levels is associated with endothelial dysfunction. It was discovered that simvastatin enhances endothelial function in patients with elevated ADMA only if the inhibitory effect of ADMA is overcome by supplemental L-arginine sustained release.

15 clinically asymptomatic, elderly subjects with elevated ADMA levels received, in a randomized order, simvastatin (40 mg/day), L-arginine sustained-release (3 g/day) prepared as described in Example 8, or a combination of both, each for 3 weeks, in a three period crossover design with at least three weeks of wash-out between treatments. Endothelium-dependent vasodilation was assessed by brachial artery ultrasound using computer-assisted image analysis; ADMA and L-arginine plasma concentrations were determined by a validated HPLC method.

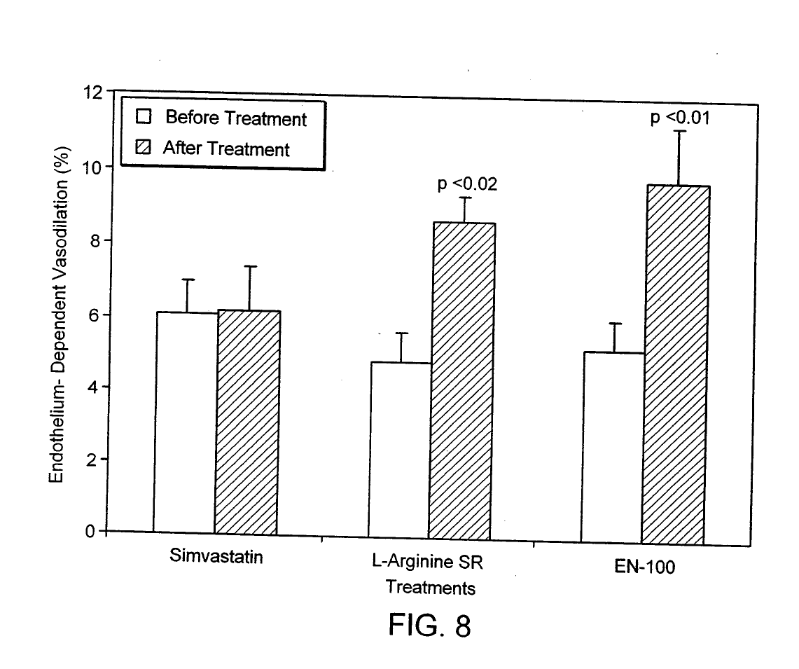

Analysis of 15 patients who completed the study revealed that both sustained release L-arginine alone or in combination with simvastatin increased percentage endothelial-dependent vasodilation, from pre-treatment measurements. The combination significantly increased the change from pre-treatment percentage endothelial-dependent vasodilation by 3.87% over that observed with simvastatin alone (p<0.025). The difference in the change in percentage endothelial-dependent vasodilation between the combination and sustained release L-arginine alone was small. Endothelium-independent vasodilation by glyceryl trinitrate was not affected by any of the treatments. L-arginine sustained release, either alone or in combination with simvastatin, significantly improved plasma L-arginine/ADMA ratio (baseline, 82.3±4.0 vs. 102.8±9.2 and 102.6±10.8, respectively, each p<0.05). These results are summarized in FIG. 8.

Simvastatin does not enhance endothelial function in subjects in whom eNOS is blocked by elevated ADMA levels; combination of simvastatin with oral L-arginine sustained release has a synergistic effect on endothelial function. As NO-mediated effects may play a major role in therapeutic effects of statins, combination with L-arginine sustained release should be considered in patients with elevated ADMA concentration.

### Example 13

### Improvement in Cholesterol Levels by Treatment with Simvastatin in Combination with L-arginine Sustained Release

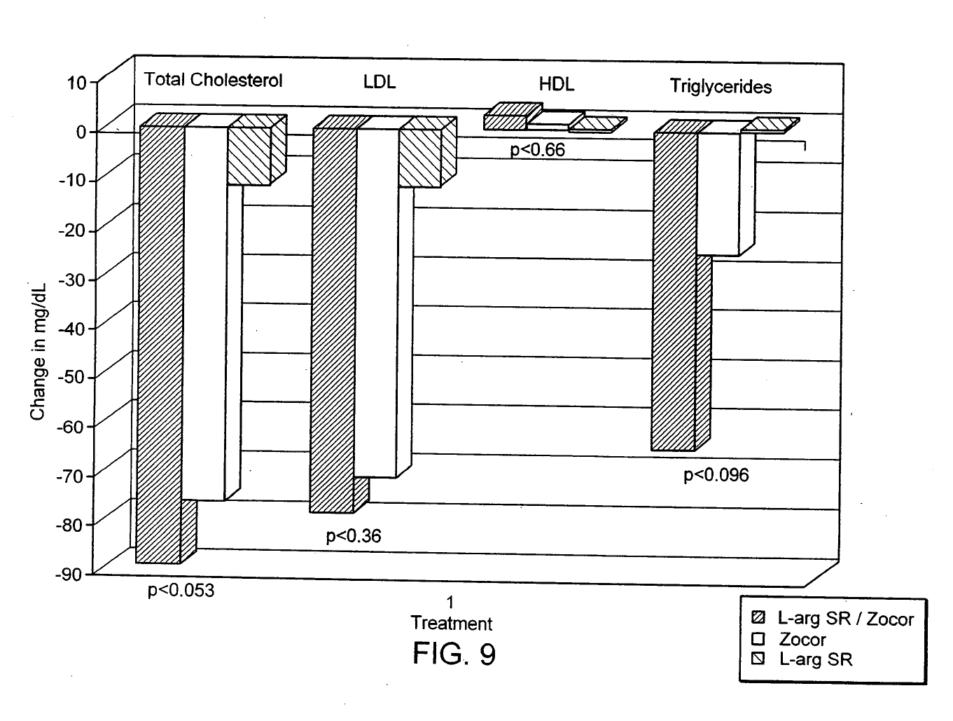

In the study described in Example 12, the change in total cholesterol (TC), LDL cholesterol, HDL cholesterol, and triglycerides was analyzed pre- and post-treatment. The results of this analysis are shown in FIG. 9. As the results demonstrate, the co-administration of sustained release L-arginine of the present invention and simvastatin lowers the total cholesterol, LDL cholesterol and triglycerides, and increases the HDL cholesterol to a greater degree than administration of simvastatin alone.

### EXAMPLE 14

### Determination of Dissolution Release of Arginine HCl in Sustained Release Arginine HCl 500 mg Tablets by HPLC

The mobile phase was prepared as follows. Initially, one liter of pH 3.3 buffer solution was prepared by weighing about 0.9 g of 1-pentanesulfonic acid sodium salt, monohydrate and 3.5 g of sodium phosphate monobasic, monohydrate into a suitable container. About 100 mL of deionized water was added to dissolve. The pH was adjusted to 3.3 by the addition of phosphoric acid. Subsequently, 850 mL of the pH 3.3 buffer was combined with 150 mL of methanol into a suitable container and mixed. The mixture was filtered through a 0.45 μm nylon membrane filter. Finally the mixture was degassed before use.

The dissolution medium (50 mM phosphate buffer at a pH of 6.8) was prepared as follows. Initially 20.0 mL of 10 M NaOH was pipetted into a 1000 mL volumetric flask and diluted with deionized water to prepare 0.2 M NaOH. Subsequently 54.44 g of Potassium Dihydrogen Phosphate, Anyhydrous was weighed into a suitable container, and dissolved and diluted with 2000 mL of deionized water. 896 mL of the 0.2 M NaOH was added to the container and diluted to 8000 mL with deionized water. Finally the mixture was degassed before use.

The dissolution sample was prepared as follows. Six Arginine HCl 500 mg tablets, prepared as described in Example 8, were weighed. Each tablet was placed in a stainless steel sinker with 900 mL of Phophate buffer (pH 6.8). The sinker was subsequently dropped into a vessel of a USP Apparatus 2 (paddle) for immediate rotation at 75 rpm at about 37° C.±0.5° C. 10 mL of the solution from the vessel was removed at 1, 2, 4, 6, 8, 10, 12 and 14 hour time points for respective dissolution analysis at each time point. Each of these samples solutions were filtered through 0.45 μm PVDF syringe filters. The filtrate was collected into HPLC vials for analysis, wherein the first 1-2 mL were discarded. Using a 10 μm Full Flow Filter, 10 mL of the dissolution medium pre-warmed to 37° C.±0.5° C. was replaced back to the dissolution vessel after every sampling point. The practitioner should be aware that the sample solution is stable up to 1 day at room temperature and is stable up to 3 days at 4° C.

The Arginine HCl standard solution was prepared as follows. 28±2 mg of Arginine HCl reference standard is accurately weighed into a 50 mL volumetric flask. The standard was dissolved in and diluted to volume with dissolution medium.

HPLC was conducted using a BDS Hypersil C18 column (5 μm, 250 mm×4.6 mm) detecting using UV at 210 nm. The column temperature was set to ambient. Generally, the run time was 9 minutes, the injection volume was 10 μL, the flow rate was 0.8 mL/min and the mobile phase was pH 3.3. Buffer/Methanol (85/15, v/v), prepared as described above.

Each trial proceeded as follows. One injection of dissolution medium followed by five consecutive injections of Arginine HCl standard solution and finally one injection of each sample solution were performed. Arginine HCl standard solution was reinjected after every six sample injections and at the end of the sequence run. The system drift throughout the run (i.e., the percent recovery of the standard solution compared to the mean of five consecutive injections of Arginine HCl standard solution) should be from about 97% to about 103%.

In determining the percent of arginine released, the practitioner must be careful to ensure that the USP trailing factor (T) for Arginine HCl peak in the injection of working standard solution is less than 2. T is calculated as follows:
T=W_{0.05}/2f
where W_{0.05} is the peak width of Arginine HCl peak at 5% of the peak height from the baseline, and f is the distance from the peak maximum to the leading edge of the peak (the distance being measured at a point 5% of the peak height from the baseline.

The percent Arginine HCl released is calculated as follows:

$$\% Release = [ ( {C}_{s} ) ( V ) ( {R}_{u} / {R}_{s} ) + {\sum }_{i = 1}^{n - 1} {C}_{i} {V}_{r} ] / ( LC )$$

where n is the total number of measurements, Vᵣ is the volume of dissolution medium for each measurement (10 mL), V is the initial volume of dissolution medium (900 mL), Cₛ is the concentration, in mg/mL, of Arginine HCl in the Working Standard Solution, Cᵢ is the concentration, in mg/mL, of Arginine HCl in each sample solution (where, i=1 to i=n−1), Rᵤ is the peak area response of Arginine HCl peak obtained from the sample solution, Rₛ is the average peak area response of Arginine HCl peak obtained from the consecutive injections of Working Standard Solution, and LC is the label claim of Arginine HCl (500 mg).

The percent released was calculated at 1, 2, 4, 6, 8, 10, 12 and 14 hours. Table VI and VIII summarize the results for various dissolution studies.

TABLE VIII
Dissolution Profiles of L-arginine SR Tablets at about 40° C./75% RH
Stability

| | Time Point | | |
|---|---|---|---|
| | Initial | 1 month | 2 months |
| Dissolution Time, hr | % Release | | |
| 0 | 0 | 0 | 0 |
| 1 | 20.4 | 21.8 | 28.1 |
| 2 | 36.4 | 36.6 | 41.1 |
| 4 | 53.5 | 54.3 | 58.5 |
| 6 | 66.8 | 67.5 | 71.5 |
| 8 | 76.6 | 77.9 | 81.3 |
| 10 | 83.1 | 85.5 | 88.3 |
| 12 | 87.2 | 89.7 | 92.9 |
| 14 | 89.1 | 92.4 | 96.0 |

### Example 15

### Simvastatin-Dependent Regulation of eNOS Expression

The following protocol was used to investigate the mechanism of the simvastatin dependent increase in eNOS function using cultured human aortic endothelial cells (HAEC) to differentiate between de novo protein synthesis versus protein mobilization or protein activation in the up regulation of eNOS function.

Human aortic endothelial cells (HAEC-c) (BioWhittaker, Walkersville, Md.) were cultured according to the following procedure. Endothelial cells in EBM-2/EGM-2 media (BioWhittaker) were grown to about 80% to about 90% confluence. Each flask of cells was washed with 5 ml media followed by the addition of 15 ml media to each cell. Cells were detached with a cell scraper and transferred to a 50 ml conical tube. Cells were pelleted by centrifugation at 800 RMP for 8 min. The supernatant was discarded and the pellet was washed with cold 1× PBS.

The cells were homogenized as follows. The pellet was loosened and 400 μl of 10× homogenization buffer (250 mM Tris at pH 7.4, 10 mM EDTA and 10 mM EGTA) was added. The sample was homogenized using a 27 G needle about 10 times. The homogenate was transferred to a 1.5 ml epindorph tube. The pellet was subsequently resuspended in 30 to 45 μl of 1× homogenization buffer.

The cells were assayed as follows. Resin slurry was prepared by washing Resin AG 50W-X8 (BioRad Laboratories, Hercules, Calif.) in an appropriate size column with 5 bed volumes of 0.5 N NaOH. The column was washed with 20 volumes of water. The resin was equilibrated with stop/equilibration buffer (50 mM NaAcetate at pH 5.5) until the eluate is within 0.05 pH units of the stop/equilibration buffer. The resulting solution is stored at 4° C. as a 50% slurry in stop/equilibration buffer. In addition, fresh 10 mM NADPH in 25 mM tris (pH 7.4) was prepared by adding 602 μl tris to a 5 mg vial of preweighed NADPH. A 1 μM Calmodulin solution was prepared by adding 0.069 mg calmodulin to 4.1 mL water. 8 μM CaCl in water was also prepared. 2× reaction buffer was prepared by combining 50 mM Tris (pH 7.4), 6 μM BH₄, 2 μM flavin adenine dinucleotide, and 2 μM flavin adenine mononucleotide. Subsequently, reaction mixture for each sample was prepared by combining 25 μl 2× reaction buffer, 5 μl 10 mM NADP, 5 μl 8 mM CaCl₂, 4 μl Calmodulin solution and 1 μl ¹⁴C Arginine. 40 μl of reaction mixture and 5 μl of sample or controls were combined in a 1.5 ml centrifuge tube. The tube was incubated for 1 hour at 37° C.

Columns were prepared by initially cutting the tip from a 1 ml pipette tip to increase the minimal diameter of the tip. 250 μl of resin slurry, prepared as described above, was pipetted into each column (Fisher Scientific, Glenlake, Ill.). The columns were washed twice with 400 μl stop/equilibration buffer (50 mM NaAcetate at pH 5.5)

Following incubation, about 400 μl of stop equilibration buffer (50 mM NaAcetate at pH 5.5) was added to each sample and control. 400 μl of this mixture is added to the equilibrated column. Each column was washed with 400 μl stop/equilibration buffer. 400 μl of column eluate was transferred to scintillation vials with 4 ml of scintillation fluid. The resulting solution is mixed well on a vortexer. A scintillation counter (Beta counter, Beckman Coulter, Inc., Fullerton, Calif.) is used to obtain the desired counts. Results are calculated, in part, by subtracting background (buffer control) from each sample. The sample values are expressed as counts per minute or as a percent of untreated cells.

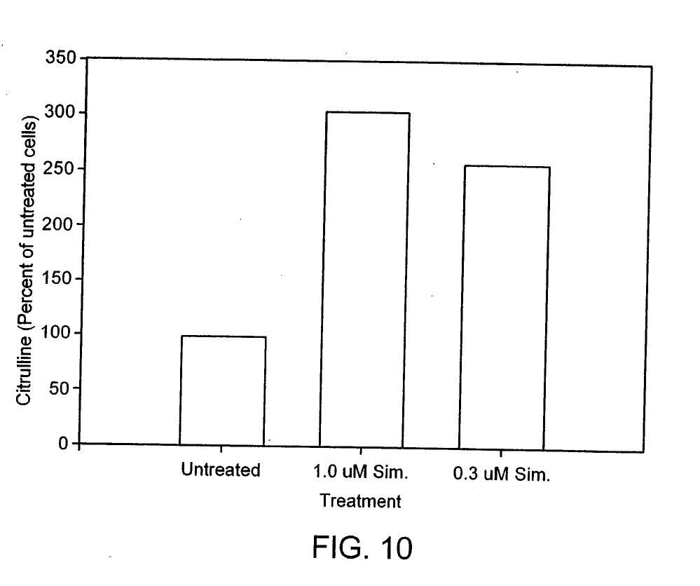

Relative eNOS function was measured by the conversion of labeled-L-arginine to L-citrulline and expressed as a percent of citrulline produced by the non-treated cells. FIG. 10 shows data from an experiment where HAEC were incubated with 1.0 or 0.3 μM simvastatin for 24 hours prior to the determination of eNOS function. Untreated cells were cultured concurrently and used to calculate relative eNOS function. FIG. 10 clearly demonstrates that simvastatin increases the level of eNOS function in cultured endothelial cells.

The collective data demonstrates that simvastatin effects eNOS expression and function in endothelial cells. A requirement for protein synthesis in the up regulation of eNOS function and the simvastatin-dependent increase in both eNOS-specific mRNA and function are consistent with a model of drug-induced modulation of eNOS gene transcription.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A sustained release L-arginine composition, comprising:
(a) about 25% to about 75% by weight of L-arginine or a pharmaceutically acceptable salt thereof;
(b) about 0.5% to about 5% by weight of polyvinylpyrrolidone;
(c) about 5% to about 40% by weight of hydroxypropyl methylcellulose;
(d) about 2% to about 20% by weight of microcrystalline cellulose;
(e) less than about 3% by weight of silicon dioxide; and
(f) less than about 3% by weight of magnesium stearate.

2. The composition of claim 1, comprising
(a) about 50% by weight of L-arginine monohydrochloride, wherein the L-arginine comprises L-arginine monohydrochloride;
(b) between about 3% and about 4% by weight of polyvinylpyrrolidone;
(c) about 35% by weight of hydroxypropyl methylcellulose;
(d) about 10% by weight of microcrystalline cellulose;
(e) less than about 1% by weight of colloidal silicon dioxide, wherein the silicon dioxide comprises colloidal silicon dioxide; and
(f) less than about 1% by weight of magnesium stearate.

3. A method for making a sustained release composition of L-arginine, comprising
(a) granulating L-arginine with a granulating agent to form granules;
(b) wet milling the granules;
(c) drying the granules;
(d) dry milling the granules; and
(e) blending the granules with at least one sustained release agent.

4. The method of claim 3, wherein the step (e) comprises the steps of pre-blending, blending and final blending the granules.

5. The method of claim 3, further comprising dry mixing the L-arginine with a binder prior to the granulating step.

6. The method of claim 5, wherein the binder comprises polyvinylpyrrolidone.

7. The method of claim 3, comprising
(a) granulating L-arginine, wherein L-arginine comprises about 50% by weight of the sustained release formulation, with granulating agent comprising polyvinylpyrrolidone, wherein polyvinylpyrrolidone comprises between about 3% and about 4% by weight of the sustained release formulation;
(b) wet milling the granules;
(c) drying the granules;
(d) dry milling the granules; and
(e) blending the granules with hydroxypropyl methylcellulose, wherein hydroxypropyl methylcellulose comprises about 35% by weight of the sustained release formulation.

8. The method of claim 9, further comprising blending the granules with microcrystalline cellulose, colloidal silicon dioxide and magnesium stearate, wherein the microcrystalline cellulose comprises about 10% by weight of the sustained release formulation, wherein colloidal silicon dioxide comprises less than about 1% of the sustained release formulation, and wherein the magnesium stearate comprises less than about 1% by weight of the sustained release formulation.

9. A food bar for use in treating or preventing a vascular disease or disorder, comprising a sustained release formulation comprising L-arginine.

10. The food bar of claim 9, wherein the sustained release formulation comprises sustained release granulars of L-arginine.

11. The food bar of claim 9, further comprising an HMG-CoA reductase inhibitor.

12. The food bar of claim 9, wherein the food bar lowers cholesterol when consumed by a subject.

13. The food bar of claim 9, wherein the food bar is for use in lowering triglyceride levels.

14. A method for increasing vasodilation in a subject, the method comprising administering sustained release L-arginine to the subject.

15. The method of claim 14, wherein the subject is administered sustained release L-arginine orally.

16. A method for lowering triglyceride levels in a subject, the method comprising administering sustained release L-arginine to the subject.

17. The method of claim 16, wherein the method lowers triglyceride levels by about 30 to about 100 mg/dL.

18. The method of claim 16, wherein the method lowers triglyceride levels by about 45 to about 75 mg/dL.

19. The method of claim 16, wherein the subject is administered sustained release L-arginine orally.

20. A method for increasing nitric oxide production in a subject with elevated asymmetrical dimethylarginine (ADMA), the method comprising administering to the subject L-arginine.

21. A method for increasing vasodilation in a subject with elevated asymmetrical dimethylarginine (ADMA), the method comprising administering to the subject release L-arginine.

22. The method of claim 20 or 21, wherein the L-arginine comprises a sustained release formulation.

23. The method of claim 20 or 21, wherein the method increases endothelial function by at least about 5 to about 15%.

24. The method of claim 20 or 21, wherein the method increases endothelial function by at least about 7 to about 12%.

25. The method of claim 20 or 21, wherein the subject has endothelial dysfunction.

26. The method of any of claims 14, 16, 20 or 21, further comprising administering to the patient an HMG-CoA reductase inhibitor.

27. The method of claim 26, wherein the HMG-CoA reductase inhibitor is simvastatin.

28. The method of any of claims 14, 16, 20 or 21, wherein the sustained release L-arginine is the composition of claim 1.
